(19) United States
(12) Patent Application Publication
(10) Pub. No.: US 2026/0224715 A1
(43) Pub. Date: Aug. 6, 2026

(54) PEPTIDES COMPRISING THIOESTER GROUPS, INCLUDING DEPSIPEPTIDE THIOESTERS, METHODS OF MAKING, AND USE THEREOF

(71) Applicant: BRANDEIS UNIVERSITY, Waltham, MA (US)

(72) Inventors: Bing XU, Newton, MA (US); Weiyi TAN, Lexington, MA (US)

(21) Appl. No.: 19/149,373

(22) PCT Filed: Jan. 31, 2024
(86) PCT No.: PCT/US24/13751

§ 371 Date: Jul. 18, 2025

Related U.S. Application Data
(60) Provisional application No. 63/442,344, filed on Jan. 31, 2023.

(51) Int. Cl. A61K 47/64; A61P 35/00; G01N 21/64

(52) U.S. Cl. CPC A61K 47/64; A61P 35/00; G01N 21/6458; G01N 21/6486

(57) ABSTRACT

The present invention relates to a compound of formula (I) or formula (II) as defined herein: Also disclosed are compositions that include compounds of formula (I) or formula (II) in an aqueous medium or a pharmaceutically acceptable carrier. Use of the compounds of formula (I) or formula (II) for delivering a drug moiety into the Golgi apparatus, imaging a cell, or treating a patient having a cancerous condition are also disclosed.

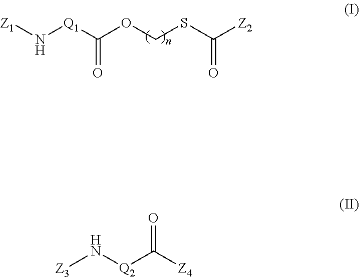

This application claims the priority benefit of U.S. Provisional Patent Application Ser. No. 63/442,344, filed Jan. 31, 2023, which is hereby incorporated by reference in its entirety.

This invention was made with government support under R01CA142746 awarded by National Institutes of Health. The government has certain rights in the invention.

### FIELD OF THE INVENTION

The present invention relates to peptides comprising thioester groups, including depsipeptide thioesters, as well as methods of making such peptides and uses thereof.

### BACKGROUND OF THE INVENTION

The Golgi apparatus (GA), a stack of flattened membrane-enclosed disks that are dynamically regulated during cell cycles in mammalian cells, is considered the “heart” of intracellular transportation (Kulkami-Gosavi et al., “Form and Function of the Golgi Apparatus: Scaffolds, Cytoskeleton and Signaling,” FEBS. Lett. 593:2289-2305 (2019); Lee et al., “Bi directional Protein Transport Between the ER and Golgi,” Annu. Rev. Cell. Dev. Biol. 20:87-123 (2004)). Increasing numbers of studies have revealed that Golgi is a hub for different signaling pathways that drive the survival and migration of cancer cells (Bivona et al., “Phospholipase Gy Activates Ras on the Golgi Apparatus by Means of RasGRP1,” Nature 424:694-698 (2003); Farber-Katz et al., “DNA Damage Triggers Golgi Dispersal Via DNA-PK and GOLPH3,” Cell 156:413-427 (2014)). Although Golgi is emerging as an important target for cancer therapy, there are, however, few approaches for targeting Golgi (Armstrong et al., “Manno-epi-cyclophellitols Enable Activity-Based Protein Profiling of Human a-Mannosidases and Discovery of New Golgi Mannosidase II Inhibitors,” J. Am. Chem. Soc. 142:13021-13029 (2020); Van Den Elsen et al., “Structure of Golgi a-mannosidase II: A Target for Inhibition of Growth and Metastasis of Cancer Cells,” EMBO J. 20:3008-3017 (2001)). While Golgi mannosidase II inhibitors are able to inhibit cancer cells, the selectivity (Dennis et al., “Growth Inhibition of Human Melanoma Tumor Xenografts in Athymic Nude Mice by Swainsonine,” Cancer Res. 50:1867-72 (1990)) or efficacy (Armstrong et al., “Manno-epi-cyclophellitols Enable Activity-Based Protein Profiling of Human a-Mannosidases and Discovery of New Golgi Mannosidase II Inhibitors,” J. Am. Chem. Soc. 142:13021-13029 (2020)) of the inhibitors remains to be improved. In addition, several studies reported the imaging of Golgi, including the commercial dyes for staining Golgi (van Echten-Deckert, et al., “1-Methylthiodihydroceramide, A Novel Analog of Dihydroceramide, Stimulates Sphinganine Degradation Resulting in Decreased De Novo Sphingolipid Biosynthesis,” J. Biol. Chem. 273:1184-91 (1998)), a smart “off-on” fluorescence probe for imaging the Golgi in cancer cells (Zhang et al., “An Off-on COX-2-specific Fluorescent Probe: Targeting the Golgi Apparatus of Cancer Cells,” J. Am. Chem. Soc. 135:11663-11669 (2013)), and carbon quantum dots localizing at Golgi (Li et al., “Chiral Nanoprobes for Targeting and Long-term Imaging of the Golgi Apparatus,” Chem. Sci. 8:6829-6835 (2017)). These imaging agents, however, require 30 minutes or longer incubation time (Zhang et al., “An Off-on COX-2-specific Fluorescent Probe: Targeting the Golgi Apparatus of Cancer Cells,” J. Am. Chem. Soc. 135:11663-11669 (2013)) or pretreatment (van Echten-Deckert, et al., “1-Methylthiodihydroceramide, A Novel Analog of Dihydroceramide, Stimulates Sphinganine Degradation Resulting in Decreased De Novo Sphingolipid Biosynthesis,” J. Biol. Chem. 273:1184-91 (1998)) and they have yet to lead to an approach for selectively inhibiting the cancer cells. Thus, there is an unmet need of fast targeting of the Golgi, particularly to inhibit cancer cells.

The present invention is directed to overcoming these and other deficiencies in the art.

### SUMMARY OF THE INVENTION

A first aspect of the invention relates to a compound of formula (I)

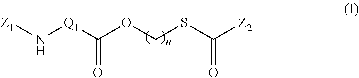

where
Z₁ is an aromatic-containing group, preferably an arylacyl; NH-Q₁-C(O) is a dipeptide containing natural or unnatural amino acids, which can be the same or different;
n is an integer from 1 to 12, such as 1 to 6, preferably 1 or 2;
Z₂ is a C₁-C₆ alkyl, C₁-C₆ alkenyl, C₁-C₆ alkynyl, —N(CH₃)₂, —CH₂OC(O)CH₃, —CH₂C(O)OCH₃,

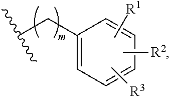

a therapeutic agent (drug moiety), or a fluorophore;
R¹, R², and R³ are independently selected at each occurrence from the group consisting of H, halogen, C₁₋₄ alkyl, —OC₁₋₄ alkyl, and —NO₂; and
m is 0, 1, 2, 3, 4, 5, or 6.

A second aspect of the invention relates to a compound of formula (II)

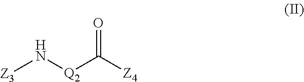

where
Z₃ is H or an aromatic-containing group, preferably an arylacyl; NH-Q₂-C(O) is a peptide containing at least three amino acid residues, including at least two aromatic amino acid residues and at least one cysteine residue, wherein the cysteine residue is optionally a modified cysteine residue having a phosphate (—PO₃H₂) group or the group —C(O)(CH₂)ₚCH₃, where p is an integer from 0 to 5, attached to the sidechain sulfur group; Z₄ is —O(CH₂)_{q}CH₃ where q is an integer from 0 to 5, a therapeutic agent (drug moiety), or a fluorophore, optionally with the group —NH(CH₂)ᵣNH— or —NH(CH₂)ᵣC(O)— between the therapeutic agent or fluorophore and the C-terminal amino acid group, where r is an integer from 0 to 5.

A third aspect of the invention relates to a composition that includes a compound according to the first or second aspect in an aqueous medium.

A fourth aspect of the invention relates to a pharmaceutical composition that includes a compound according to the first or second aspect in a pharmaceutically acceptable carrier.

A fifth aspect of the invention relates to a method of delivering a drug moiety into the Golgi apparatus. This method includes the steps of providing a compound according to the first or second aspect, wherein Z₂ or Z₄ is a drug moiety, or a composition according to the third or fourth aspect; and contacting a cell with the compound or the composition, whereby the compound is taken up by the cell and targeted to the Golgi apparatus within the cell.

A sixth aspect of the invention relates to a method treating a patient having a cancerous condition. This method includes administering a pharmaceutical composition according to the fourth aspect to a patient having a cancerous condition, wherein said administering is effective to inhibit cancer cell survival.

A seventh aspect of the invention relates to a method of imaging a cell. This method includes the steps of providing a compound according to the first aspect, wherein Z₁ or Z₂ comprises a fluorophore or the dipeptide comprises a non-natural amino acid comprising a fluorophore moiety, or a composition comprising the compound; contacting a cell with the compound or the composition, whereby the compound is taken up by the cell and targeted to the Golgi apparatus within the cell; and obtaining an image of the cell, whereby the Golgi apparatus is identified by fluorescence from the fluorophore.

The accompanying examples demonstrate the development two different approaches for targeting the Golgi apparatus. One approach utilizes depsipeptide thioesters and the other approach involves longer cysteine-containing peptides where the cysteine residue includes an S-thioester or-thiophosphate group on the sidechain. The depsipeptide thioesters contain both carboxylester and thioester bonds for linking functional motifs, such as fluorophores, to the peptides. The ability of the depsipeptide thioesters for imaging Golgi of cells is examined with the concentration from 50 nanomolar to 1 micromolar. The depsipeptide thioesters were found to be able to inhibit the growth of cancer cells with the IC₅₀ from submicromolar to tens of micromolar. The cysteine-containing peptides exhibit a decent Golgi-targeting ability as well as a promising anticancer performance. The use of natural amino acid as the source of sulfhydryl groups makes the synthesis of the Golgi targeting peptides easier.

The depsipeptide thioesters enter cells and form assemblies at Golgi much faster than the previously reported thiophosphopeptides (Tan et al., “Enzymatic Assemblies of Thiophosphopeptides Instantly Target Golgi Apparatus and Selectively Kill Cancer Cells,” Angewandte Chemie Internat'l Ed. 60(23):12796-12801 (2021), which is hereby incorporated by reference in its entirety) and peptide thioesters (Tan et al., “Enzyme-responsive Peptide Thioesters for Targeting Golgi Apparatus,” J Am. Chem. Soc. 144(15):6709-6713 (2022), which is hereby incorporated by reference in its entirety) at low concentrations, such as nanomolar concentration. Contrasting to the peptide thioesters, changing the self-assembly motif, diphenylalanine, to other neutral dipeptide still maintains the Golgi-targeting ability, suggesting a general molecular architecture for Golgi targeting. In addition, these depsipeptide thioesters show cell selectivity due to the different expression level of carboxylesterase, which can hydrolyze the ester bond and lead to cell survival.

### BRIEF DESCRIPTION OF THE DRAWINGS

FIG. 1 shows overlapping of the green fluorescence from depsipeptide thioester ([1]=1 μM) with red fluorescence from Golgi-RFP transfected HeLa cells.

FIG. 2 shows confocal laser scanning microscopy (CLSM) images of different cancer cell lines treated with 1 (500 nM, 30 min).

FIG. 3 shows CLSM images of HeLa cells treated with 1 (500 nM) for 1, 4, 8, 16, 24, 32 min.

FIG. 4 shows cytotoxicity of BPffesSAc (2) against HeLa cells for 24 h.

FIG. 5 shows cytotoxicity of BPffesSAc (2) against B16F10 cells for 24 h.

FIG. 6 shows cytotoxicity of BPffesSAc (2) against NIH3 T3 cells for 24 h.

FIG. 7 shows cytotoxicity of BPffesSAc (2) against NIH3 T3 cells for 24 h.

FIG. 8 shows cytotoxicity of BPffesSAc (2) against A431 cells for 24 h.

FIG. 9 shows cytotoxicity of BPffesSAc (2) against KPCA-B cells for 24 h.

FIG. 10 shows cytotoxicity of BPffesSAc (2) against KPCA-C cells for 24 h.

FIG. 11 shows cytotoxicity of BPffesSAc (2) against SKOV-3 cells for 24 h.

FIG. 12 shows cytotoxicity of BPffesSAc (2) against OVCAR-4 cells for 24 h.

### DETAILED DESCRIPTION OF THE INVENTION

As used herein and in the appended claims, the singular “a”, “an” and “the” include the plural reference unless the context clearly dictates otherwise. Thus, for example, reference to a “depsipeptide thioester compound” or cysteine-containing peptide includes a plurality of such compounds or peptides.

One aspect of the invention relates to a depsipeptide thioester compound comprising the structure according to formula (I):

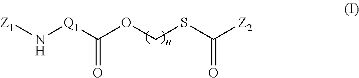

where
Z₁ is an aromatic-containing group, preferably an arylacyl;
NH-Q₁-C(O) is a dipeptide containing natural or unnatural amino acids, which can be the same or different;
n is an integer from 1 to 12, such as 1 to 6, preferably 1 or 2;
Z₂ is a C₁-C₆ alkyl, C₁-C₆ alkenyl, C₁-C₆ alkynyl, —N(CH₃)₂, —CH₂OC(O)CH₃, —CH₂C(O)OCH₃,

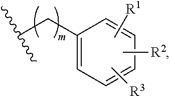

a therapeutic agent (drug moiety), or a fluorophore;
R¹, R², and R³ are independently selected at each occurrence from the group consisting of H, halogen, C₁₋₄ alkyl, —OC₁₋₄ alkyl, and —NO₂; and
m is 0, 1, 2, 3, 4, 5, or 6.

In some embodiments, Z₂ is

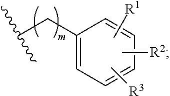

R¹, R², and R³ are independently selected at each occurrence from the group consisting of H, F, Cl, Me, Et, Pr, i-Pr, t-Bu, —OMe, —OEt, and —NO₂; and m is 0, 1, or 2. In certain embodiments, all of R¹, R², and R³ are H. In certain embodiments, one of R¹, R², and R³ is other than H. In other embodiments, two of R¹, R², and R³ are other than H. In another embodiment, all three of R¹, R², and R³ are other than H. When R¹, R², and R³ are all H and m is 0, then Z₂ is an unsubstituted phenyl group.

As discussed more fully below, the depsipeptide thioester compound may be capable of forming nanoparticles in an aqueous medium, and are capable of cellular uptake and targeting to the Golgi apparatus.

The term “amino acid” includes analogues, derivatives, and congeners of any specific amino acid referred to herein. Furthermore, the term “amino acid” includes both D-and L-amino acids. Hence, an amino acid which is identified herein by its name, three letter or one letter symbol and is not identified specifically as having the D or L configuration, is understood to assume any one of the D or L configurations. For example, 2-Nal or 2-nal refer to the L and D configurations, respectively, of the analogue 3-(2-naphthyl)-alanine.

Naturally occurring amino acids are identified throughout by the conventional three-letter and/or one-letter abbreviations, corresponding to the trivial name of the amino acid, in accordance with the following list: Alanine (Ala), Arginine (Arg), Asparagine (Asn), Aspartic acid (Asp), Cysteine (Cys), Glutamic acid (Glu), Glutamine (Gin), Glycine (Gly), Histidine (His), Isoleucine (lie), Leucine (Leu), Lysine (Lys), Methionine (Met), Phenylalanine (Phe), Proline (Pro), Serine (Ser), Threonine (Thr), Tryptophan (Trp), Tyrosine (Tyr), and Valine (Val). The abbreviations are accepted in the peptide art and are recommended by the IUPAC-IUB commission in biochemical nomenclature.

Preferred amino acids in the dipeptide include, without limitation, L- and D-amino acids selected from: Phe, phe, Val, val, Gly, gly, Leu, leu, Met, met, Tyr, tyr, Ile, ile, Ala, ala, 2-Nal, 2-nal, Leu, leu, Dmt (2,6-dimethyl-L-tyrosine), dmt, I-Phe (4-iodo-phenylalanine), i-phe, and dap(NBD) [3-N-(7-nitrobenz-2-oxa-1,3-diazole-4-yl)-2,3-diaminopropionic acid]. The structures of I-Phe and dap(NBD) are shown below:

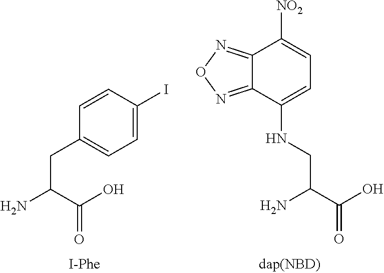

In certain embodiments of the invention, the dipeptide contains only D-amino acids. In an alternative embodiment, the dipeptide contains only L-amino acids, or a mixture of L- and D-amino acids. The amino acids in the dipeptide can be the same or different.

Exemplary dipeptides include, without limitation, Phe-Phe, Leu-Leu, Ile-Ile, Val-Val, Met-Met, Ala-Ala, Gly-Gly, Tyr-Tyr, photo-Met-Leu, I-Phe-I-Phe, and dap(NBD)-Phe, including both L- and D-amino acid variants thereof. Specific exemplary dipeptides include, without limitation, D-Phe-D-Phe, D-Leu-D-Leu, D-Ile-D-Ile, D-Val-D-Val, D-Met-D-Met, D-Ala-D-Ala, D-Gly-D-Gly, D-Tyr-D-Tyr, L-photo-Met-L-Leu, D-I-Phe-D-I-Phe, and D-dap(NBD)-D-Phe.

As noted above, Z₁ is an aromatic-containing group, preferably an arylacyl that contains a single-ring, multiple ring, or fused-ring aryl groups, optionally containing one or more heteroatoms. Exemplary aromatic-containing groups (Z₁) include, without limitation,

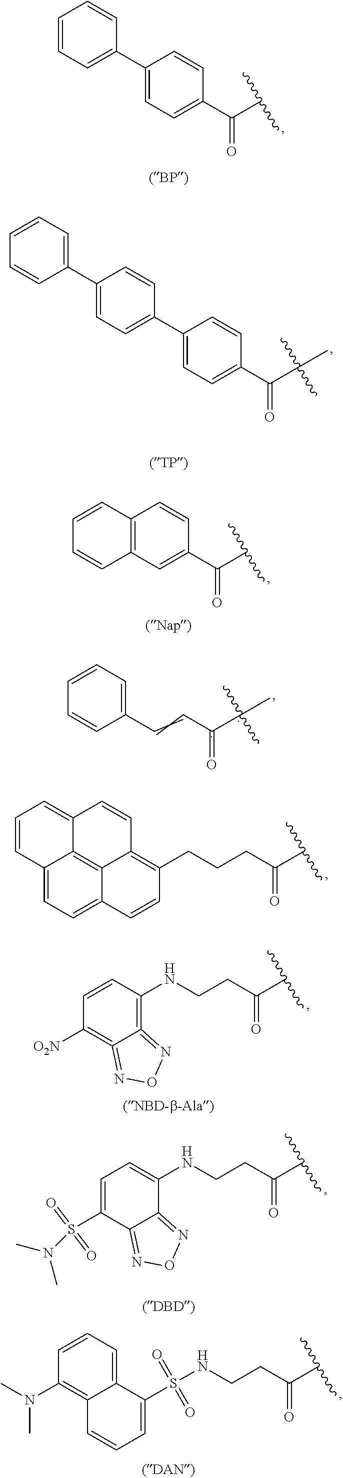

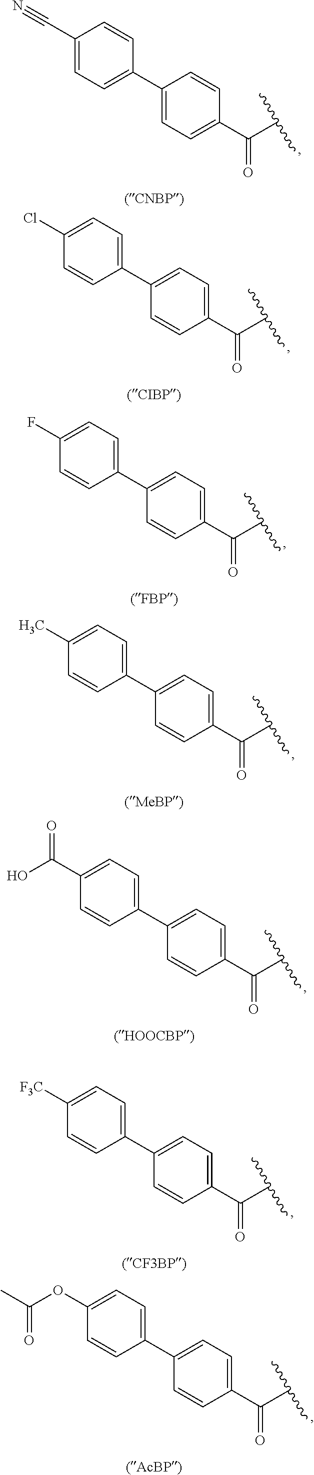

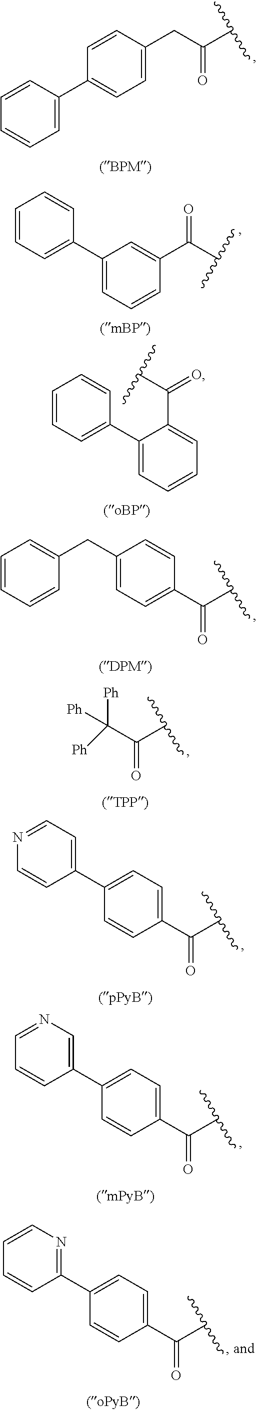

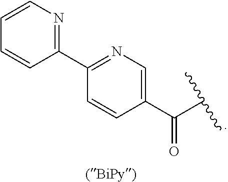 [SMILES: *C(=O)c1ccc(-c2ccccn2)nc1]

Exemplary Z₂ groups include, without limitation, C₁-C₆ alkyl groups such as methyl, ethyl, propyl, isopropyl, butyl, isobutyl, t-butyl, pentyl, 2-methyl-pentyl, 3-methyl-pentyl, and hexyl; C₁-C₆ alkenyl groups such as vinyl, 1-propenyl, 2-propenyl, 1-butenyl, 2-butenyl, 3-butenyl, 1-pentenyl, 2-pentenyl, 1-hexenyl, and 2-hexenyl; C₁-C₆ alkynyl groups such as propynyl, 1-butynyl, 3-butynyl, and 4-pentynyl; —N(CH₃)₂; —CH₂OC(O)CH₃; —CH₂C(O)OCH₃; and unsubstituted phenyl.

Additional exemplary Z₂ groups also include, without limitation, the following substituted phenyl groups where m=0 or 1:

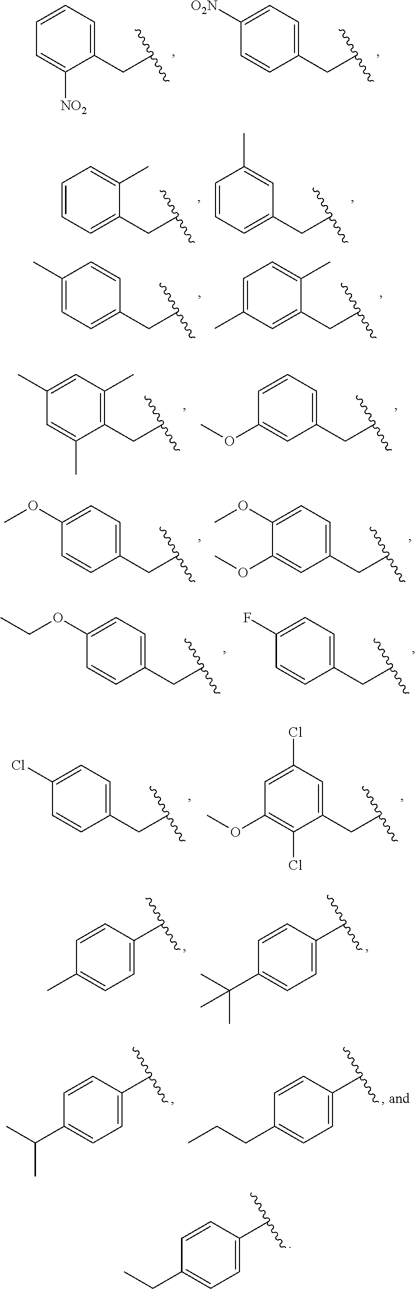

Additional substituted phenyl or benzyl groups can be obtained using commercially available substituted phenylacetyl chloride or substituted benzoyl chloride reactants.

Additional Z₂ groups include therapeutic agents such as antioxidants, coenzymes, vitamins, metabolites, analgesics, anti-inflammatory agents, antihelminthics, anti-arrhythmic agents, antibacterial agents, anti-viral agents, anti-coagulants, anti-depressants, anti-diabetics, anti-epileptics, anti-fungal agents, anti-gout agents, anti-hypertensive agents, anti-thrombogenic agents, anti-claudication agents, anti-atherosclerotic drugs, vascular agents, anti-malarials, anti-migraine agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, b-blockers, cardiac inotropic agents, corticosteroids, diuretics, anti-parkinsonian agents, gastro-intestinal agents, histamine receptor antagonists, keratolyptics, lipid regulating agents, anti-anginal agents, Cox-2 inhibitors, leukotriene inhibitors, macrolides, muscle relaxants, nutritional agents, opioid analgesics, protease inhibitors, sex hormones, stimulants, anti-osteoporosis agents, anti-obesity agents, cognition enhancers, anti-urinary incontinence agents, anti-benign prostate hypertrophy agents, essential fatty acids, non-essential fatty acids, cytokines, growth factors, antibodies, radioprotective agents, and cardioprotective agents. Further non-limiting examples of the therapeutic agents in the pharmaceutical composition include: acetretin, albendazole, albuterol, aminoglutethimide, amiodarone, amlodipine, amphetamine, amphotericin B, arginine, atorvastatin, atovaquone, azithromycin, baclofen, beclomethasone, benazepril, benzonatate, betamethasone, bicalutanide, budesonide, bupropion, busulfan, butenafme, calcifediol, calcipotriene, calcitriol, camptothecin, candesartan, capsaicin, captopril, carbamezepine, carotenes, celecoxib, cerivastatin, cetirizine, chlorpheniramine, cholecalciferol, cilazepril, cilostazol, cimetidine, cinnarizine, ciprofloxacin, cisapride, clarithromycin, clemastine, clomiphene, clomipramine, clonidine, clopidogrel, codeine, coenzyme Q10, cyclobenzaprine, cyclosporin, danazol, dantrolene, dexchlorpheniramine, diclofenac, dicoumarol, digoxin, dehydroepiandrosterone, dihydroergotamine, dihydrotachysterol, dirithromycin, donezepil, doxazosin, efavirenz, eprosartan, ergocalciferol, ergotamine, essential fatty acid sources, etodolac, etoposide, famotidine, fenofibrate, fentanyl, fexofenadine, finasteride, fluconazole, flurbiprofen, fluvastatin, fosphenyloin, frovatriptan, fuirazolidone, gabapentin, gemfibrozil, glibenclamide, glipizide, glyburide, glimepiride, griseofulvin, halofantrine, ibuprofen, irbesartan, irinotecan, isosorbide dinitrate, isotretinoin, itraconazole, ivermectin, ketenserin, ketoconazole, ketorolac, lamotrigine, lansoprazole, leflunomide, lisinopril, loperamide, loratadine, losartan, lovastatin, L-thryroxine, lutein, lycopene, medroxyprogesterone, mifepristone, mefloquine, megestrol acetate, methadone, methoxsalen, methyldopa, metronidazole, miconazole, midazolam, miglitol, minoxidil, mitoxantrone, montelukast, moxonidine, nabumetone, nalbuphine, naratriptan, nelfmavir, nifedipine, nil solidipine, nilutanide, nitrofurantoin, nitroglycerin, nizatidine, omeprazole, oprevelkin, oestradiol, oxaprozin, paclitaxel, paracalcitol, paroxetine, pentazocine, pioglitazone, pizofetin, prazosin, pravastatin, prednisolone, probucol, progesterone, pseudoephedrine, pyridostigmine, rabeprazole, raloxifene, rofecoxib, repaglinide, rifabutine, rifapentine, rimexolone, ritanovir, rizatriptan, rosiglitazone, saquinavir, sertraline, sibutramine, sildenafil citrate, simvastatin, sirolimus, spironolactone, sumatriptan, tacrine, tacrolimus, tamoxifen, tamsulosin, targretin, tazarotene, telmisartan, teniposide, terbinafme, terazosin, tetrahydrocannabinol, tiagabine, ticlopidine, tirofibran, tizanidine, topiramate, topotecan, toremitfene, tramadol, tretinoin, troglitazone, trovafloxacin, ubidecarenone, urapidil, valsartan, venlafaxine, verteporfm, vigabatrin, vitamin A, vitamin D, vitamin E, vitamin K, zafirlukast, zileuton, zolmitriptan, zolpidem, zopiclone, pharmaceutically acceptable salts, isomers, and derivatives thereof, and mixtures thereof.

Additional Z₂ groups include fluorophores such as 4-nitro-2,1,3-benzoxadiazolyl (NBD), 5-(dimethylamino)naphthalene-1-sulfonyl, 4-(N,N-dimethylaminosulfonyl)-2,1,3-benzoxadiazolyl, and 9-acridinyl. NBD is exemplified in the accompanying examples. 5-(dimethylamino)naphthalene-1-sulfonyl chloride is a reagent that reacts with primary amino groups in both aliphatic and aromatic amines to produce stable blue-or blue-green-fluorescent sulfonamide adducts. 4-(N,N-Dimethylaminosulfonyl)-7-isothiocyanato-2,1,3-benzoxadiazole can be used to react with primary amines to afford a stable and strong fluorophore (Matsunaga et al., Anal. Chem. 67(23):4276-82 (1995), which is hereby incorporated by reference in its entirety). 9-acridinyl carboxylic acid can be used to label primary amines in peptides (Carlson et al., Org. Lett. 2(10): 1465-1468 (2000), which is hereby incorporated by reference in its entirety).

Exemplary depsipeptide thioester compounds according to formula (I) include, without limitation:

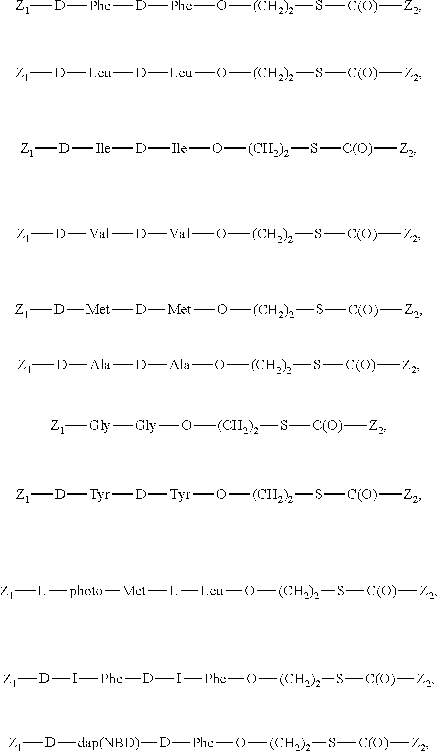

where Z₁ and Z₂ are defined above. In certain embodiments, Z₂ is —CH₃, —CH₂CH₃, —N(CH₃)₂, —CH₂OC(O)CH₃, —CH₂C(O)OCH₃, or phenyl or any one of the substituted phenyl groups as noted above. It is contemplated that each of these Z₂ groups can be present for each of the exemplary Z₁ groups noted above.

Another aspect of the invention relates to a cysteine-containing peptide comprising the structure according to formula (II):

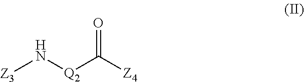

where
Z₃ is H or an aromatic-containing group, preferably an arylacyl; NH-Q₂-C(O) is a peptide containing at least three amino acid residues, preferably three to sixteen amino acid residues, three to fourteen amino acid residues, three to twelve amino acid residues, or three to ten amino acid residues (such as 3, 4, 5, 6, 7, 8, 9, or 10 amino acid residues) including at least two aromatic amino acid residues and at least one cysteine residue, wherein the cysteine residue is optionally a modified cysteine residue having a phosphate (—PO₃H₂) group or the group —C(O)(CH₂)ₚCH₃, where p is an integer from 0 to 5, attached to the sidechain sulfur group;
Z₄ is —O(CH₂)_{q}CH₃ where q is an integer from 0 to 5, a therapeutic agent (drug moiety), or a fluorophore, optionally with the group —NH(CH₂)ᵣNH— or —NH(CH₂)ᵣC(O)— between the therapeutic agent or fluorophore and the C-terminal amino acid group, where r is an integer from 0 to 5.

Z₃ can be any of the groups defined as Z₁, or H.

The therapeutic agents or fluorophores identified above for Z₂ can also be used as Z₄ in the compounds of formula (II).

Suitable aromatic amino acids present in the peptide include, without limitation, tyrosine, phenylalanine, 2-naphthylalanine, 1-naphthylalanine, biphenylalanine, 4-(2-phenylethyl)phenylalanine, and 4-(2-phenylethenyl)phenylalanine.

In some embodiments, an unmodified cysteine is present in the peptide.

In other embodiments, a modified cysteine residue is present in the peptide. In certain embodiments the modified cysteine residue comprises the group —C(O)(CH₂)ₚCH₃ attached to the terminal sulfur group of the cysteine sidechain, where p=0, 1, or 2. In certain embodiments, more than one cysteine residue can be present in the peptide, and where two or more cysteine residues are present the two or more cysteine residues can all have modified sidechains bearing —C(O)(CH₂)ₚCH₃ attached to the terminal sulfur group (as defined above) or only one of the multiple cysteine residues can be so-modified.

In other embodiments, the modified cysteine residue comprises a phosphate (—PO₃H₂) group attached to the terminal sulfur group of the cysteine sidechain. In certain embodiments, more than one cysteine residue can be present in the peptide, and where two or more cysteine residues are present the two or more cysteine residues can all have modified sidechains bearing phosphate groups or a mixture of phosphate groups and —C(O)(CH₂)ₚCH₃ groups attached to the terminal sulfur group (as defined above). Alternatively, only one of the multiple cysteine residues can be so-modified with a phosphate group.

In certain embodiments of the invention, the polypeptide contains only D-amino acids. In an alternative embodiment, the polypeptide contains only L-amino acids, or a mixture of L- and D-amino acids. The amino acids in the polypeptide can be the same or different.

Exemplary polypeptides present in the compounds of formula (II) include, without limitation, Cys-Phe-Phe, Cys-bPhe-bPhe, Cys-Tyr-Tyr, Cys-(1-Nal)-(1-Nal), Cys-(2-Nal)-(2-Nal), Cys-(4-(2-phenylethyl)Phe-(4-(2-phenylethyl)Phe, Cys-(4-(2-phenylethenyl)Phe-(4-(2-phenylethenyl)Phe, Phe-Phe-Cys, Tyr-Tyr-Cys, (1-Nal)-(1-Nal)-Cys, (2-Nal)-(2-Nal)-Cys, (4-(2-phenylethyl)Phe-(4-(2-phenylethyl)Phe-Cys, (4-(2-phenylethenyl)Phe-(4-(2-phenylethenyl)Phe-Cys, or bPhe-bPhe-Cys.

Exemplary cysteine-containing peptides according to formula (II) include, without limitation:
Z₃-L-(S-acetyl)Cys-L-bPhe-L-bPhe-Z₄,
Z₃-D-(S-acetyl)Cys-D-bPhe-D-bPhe-Z₄,
Z₃-L-(S-acetyl)Cys-L-Phe-L-Phe-Z₄,
Z₃-D-(S-acetyl)Cys-D-Phe-D-Phe-Z₄,
Z₃-L-(S-acetyl)Cys-L-Tyr-L-Tyr-Z₄,
Z₃-D-(S-acetyl)Cys-D-Tyr-D-Tyr-Z₄,
Z₃-L-(S-acetyl)Cys-L-(1-Nal)-L-(1-Nal)-Z₄,
Z₃-D-(S-acetyl)Cys-D-(1-Nal)-D-(1-Nal)-Z₄,
Z₃-L-(S-acetyl)Cys-L-(2-Nal)-L-(2-Nal)-Z₄,
Z₃-D-(S-acetyl)Cys-D-(2-Nal)-D-(2-Nal)-Z₄,
Z₃-L-(S-acetyl)Cys-L-(4-(2-phenylethyl)Phe-L-(4-(2-phenylethyl)Phe-Z₄,
Z₃-D-(S-acetyl)Cys-D-(4-(2-phenylethyl)Phe-D-(4-(2-phenylethyl)Phe-Z₄,
Z₃-L-(S-acetyl)Cys-L-(4-(2-phenylethenyl)Phe-L-(4-(2-phenylethenyl)Phe-Z₄,
Z₃-D-(S-acetyl)Cys-D-(4-(2-phenylethenyl)Phe-D-(4-(2-phenylethenyl)Phe-Z₄,
Z₃-L-(S—PO₃H₂)Cys-L-bPhe-L-bPhe-Z₄,
Z₃-D-(S—PO₃H₂)Cys-D-bPhe-D-bPhe-Z₄,
Z₃-L-(S—PO₃H₂)Cys-L-Phe-L-Phe-Z₄,
Z₃-D-(S—PO₃H₂)Cys-D-Phe-D-Phe-Z₄,
Z₃-L-(S—PO₃H₂)Cys-L-Tyr-L-Tyr-Z₄,
Z₃-D-(S—PO₃H₂)Cys-D-Tyr-D-Tyr-Z₄,
Z₃-L-(S—PO₃H₂)Cys-L-(1-Nal)-L-(1-Nal)-Z₄,
Z₃-D-(S—PO₃H₂)Cys-D-(1-Nal)-D-(1-Nal)-Z₄,
Z₃-L-(S—PO₃H₂)Cys-L-(2-Nal)-L-(2-Nal)-Z₄,
Z₃-D-(S—PO₃H₂)Cys-D-(2-Nal)-D-(2-Nal)-Z₄,
Z₃-L-(S—PO₃H₂)Cys-L-(4-(2-phenylethyl)Phe-L-(4-(2-phenylethyl)Phe-Z₄,
Z₃-D-(S—PO₃H₂)Cys-D-(4-(2-phenylethyl)Phe-D-(4-(2-phenylethyl)Phe-Z₄,
Z₃-L-(S—PO₃H₂)Cys-L-(4-(2-phenylethenyl)Phe-L-(4-(2-phenylethenyl)Phe-Z₄,
Z₃-D-(S—PO₃H₂)Cys-D-(4-(2-phenylethenyl)Phe-D-(4-(2-phenylethenyl)Phe-Z₄,
Z₃-L-Cys-L-bPhe-L-bPhe-Z₄,
Z₃-D-Cys-D-bPhe-D-bPhe-Z₄,
Z₃-L-Cys-L-Phe-L-Phe-Z₄,
Z₃-D-Cys-D-Phe-D-Phe-Z₄,
Z₃-L-Cys-L-Tyr-L-Tyr-Z₄,
Z₃-D-Cys-D-Tyr-D-Tyr-Z₄,
Z₃-L-Cys-L-(1-Nal)-L-(1-Nal)-Z₄,
Z₃-D-Cys-D-(1-Nal)-D-(1-Nal)-Z₄,
Z₃-L-Cys-L-(2-Nal)-L-(2-Nal)-Z₄,
Z₃-D-Cys-D-(2-Nal)-D-(2-Nal)-Z₄,
Z₃-L-Cys-L-(4-(2-phenylethyl)Phe-L-(4-(2-phenylethyl)Phe-Z₄,
Z₃-D-Cys-D-(4-(2-phenylethyl)Phe-D-(4-(2-phenylethyl)Phe-Z₄,
Z₃-L-Cys-L-(4-(2-phenylethenyl)Phe-L-(4-(2-phenylethenyl)Phe-Z₄,
Z₃-D-Cys-D-(4-(2-phenylethenyl)Phe-D-(4-(2-phenylethenyl)Phe-Z₄,
Z₃-L-bPhe-L-bPhe-L-(S-acetyl)Cys-Z₄,
Z₃-D-bPhe-D-bPhe-D-(S-acetyl)Cys-Z₄,
Z₃-L-Phe-L-Phe-L-(S-acetyl)Cys-Z₄,
Z₃-D-Phe-D-Phe-D-(S-acetyl)Cys-Z₄,
Z₃-L-Tyr-L-Tyr-L-(S-acetyl)Cys-Z₄,
Z₃-D-Tyr-D-Tyr-D-(S-acetyl)Cys-Z₄,
Z₃-L-(1-Nal)-L-(1-Nal)-L-(S-acetyl)Cys-Z₄,
Z₃-D-(1-Nal)-D-(1-Nal)-D-(S-acetyl)Cys-Z₄,
Z₃-L-(2-Nal)-L-(2-Nal)-L-(S-acetyl)Cys-Z₄,
Z₃-D-(2-Nal)-D-(2-Nal)-D-(S-acetyl)Cys-Z₄,
Z₃-L-(4-(2-phenylethyl)Phe-L-(4-(2-phenylethyl)Phe-L-(S-acetyl)Cys-Z₄,
Z₃-D-(4-(2-phenylethyl)Phe-D-(4-(2-phenylethyl)Phe-D-(S-acetyl)Cys-Z₄,
Z₃-L-(4-(2-phenylethenyl)Phe-L-(4-(2-phenylethenyl)Phe-L-(S-acetyl)Cys-Z₄,
Z₃-D-(4-(2-phenylethenyl)Phe-D-(4-(2-phenylethenyl)Phe-D-(S-acetyl)Cys-Z₄,
Z₃-L-bPhe-L-bPhe-L-Cys-Z₄,
Z₃-D-bPhe-D-bPhe-D-Cys-Z₄,
Z₃-L-Phe-L-Phe-L-Cys-Z₄,
Z₃-D-Phe-D-Phe-D-Cys-Z₄,
Z₃-L-Tyr-L-Tyr-L-Cys-Z₄,
Z₃-D-Tyr-D-Tyr-D-Cys-Z₄,
Z₃-L-(1-Nal)-L-(1-Nal)-L-Cys-Z₄,
Z₃-D-(1-Nal)-D-(1-Nal)-D-Cys-Z₄,
Z₃-L-(2-Nal)-L-(2-Nal)-L-Cys-Z₄,
Z₃-D-(2-Nal)-D-(2-Nal)-D-Cys-Z₄,
Z₃-L-(4-(2-phenylethyl)Phe-L-(4-(2-phenylethyl)Phe-L-Cys-Z₄,
Z₃-D-(4-(2-phenylethyl)Phe-D-(4-(2-phenylethyl)Phe-D-Cys-Z₄,
Z₃-L-(4-(2-phenylethenyl)Phe-L-(4-(2-phenylethenyl)Phe-L-Cys-Z₄,
Z₃-D-(4-(2-phenylethenyl)Phe-D-(4-(2-phenylethenyl)Phe-D-Cys-Z₄,
Z₃-L-bPhe-L-bPhe-L-(S—PO₃H₂)Cys-Z₄,
Z₃-D-bPhe-D-bPhe-D-(S—PO₃H₂)Cys-Z₄,
Z₃-L-Phe-L-Phe-L-(S—PO₃H₂)Cys-Z₄,
Z₃-D-Phe-D-Phe-D-(S—PO₃H₂)Cys-Z₄,
Z₃-L-Tyr-L-Tyr-L-(S—PO₃H₂)Cys-Z₄,
Z₃-D-Tyr-D-Tyr-D-(S—PO₃H₂)Cys-Z₄,
Z₃-L-(1-Nal)-L-(1-Nal)-L-(S—PO₃H₂)Cys-Z₄,
Z₃-D-(1-Nal)-D-(1-Nal)-D-(S—PO₃H₂)Cys-Z₄,
Z₃-L-(2-Nal)-L-(2-Nal)-L-(S—PO₃H₂)Cys-Z₄,
Z₃-D-(2-Nal)-D-(2-Nal)-D-(S—PO₃H₂)Cys-Z₄,
Z₃-L-(4-(2-phenylethyl)Phe-L-(4-(2-phenylethyl)Phe-L-(S—PO₃H₂)Cys-Z₄,
Z₃-D-(4-(2-phenylethyl)Phe-D-(4-(2-phenylethyl)Phe-D-(S—PO₃H₂)Cys-Z₄,
Z₃-L-(4-(2-phenylethenyl)Phe-L-(4-(2-phenylethenyl)Phe-L-(S—PO₃H₂)Cys-Z₄, or
Z₃-D-(4-(2-phenylethenyl)Phe-D-(4-(2-phenylethenyl)Phe-D-(S—PO₃H₂)Cys-Z₄,
   where Z₃ is independently H— or NBD-β-Ala, and Z₄ is independently —OCH₃ or —NH(CH₂)₂—NBD.

In certain embodiments, the compound of formula (II) is selected from the following:

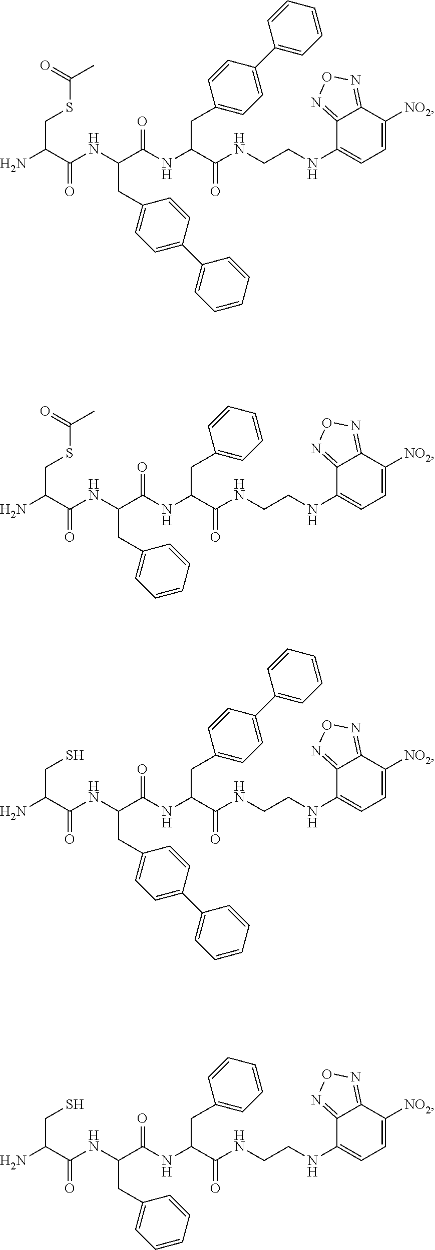 [SMILES: CC(=O)SCC(N)C(=O)NC(Cc1ccc(-c2ccccc2)cc1)C(=O)NC(Cc1ccc(-c2ccccc2)cc1)C(=O)NCCNc1ccc([N+](=O)[O-])c2nonc12 ; CC(=O)SCC(N)C(=O)NC(Cc1ccccc1)C(=O)NC(Cc1ccccc1)C(=O)NCCNc1ccc([N+](=O)[O-])c2nonc12 ; NC(CS)C(=O)NC(Cc1ccc(-c2ccccc2)cc1)C(=O)NC(Cc1ccc(-c2ccccc2)cc1)C(=O)NCCNc1ccc([N+](=O)[O-])c2nonc12 ; NC(CS)C(=O)NC(Cc1ccccc1)C(=O)NC(Cc1ccccc1)C(=O)NCCNc1ccc([N+](=O)[O-])c2nonc12]

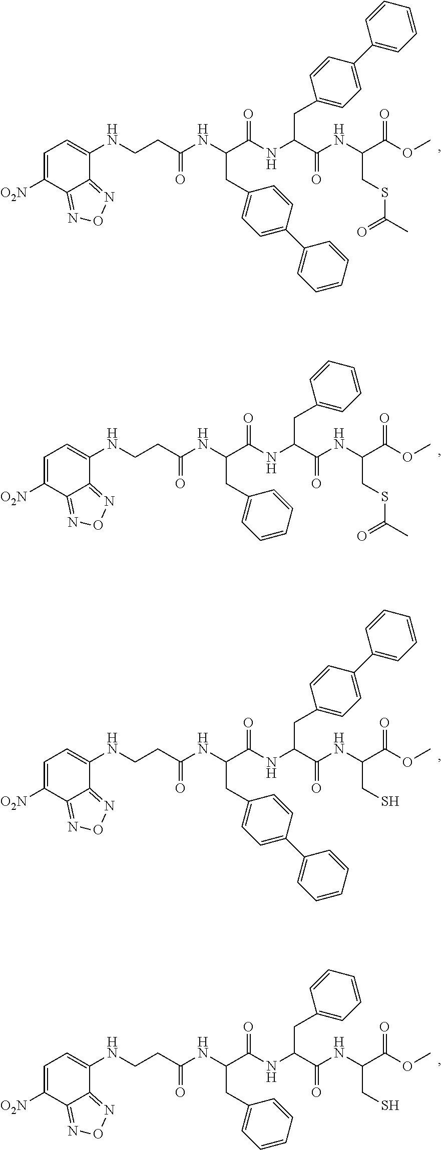 [SMILES: COC(=O)C(CSC(C)=O)NC(=O)C(Cc1ccc(-c2ccccc2)cc1)NC(=O)C(Cc1ccc(-c2ccccc2)cc1)NC(=O)CCNc1ccc([N+](=O)[O-])c2nonc12 ; COC(=O)C(CSC(C)=O)NC(=O)C(Cc1ccccc1)NC(=O)C(Cc1ccccc1)NC(=O)CCNc1ccc([N+](=O)[O-])c2nonc12 ; COC(=O)C(CS)NC(=O)C(Cc1ccc(-c2ccccc2)cc1)NC(=O)C(Cc1ccc(-c2ccccc2)cc1)NC(=O)CCNc1ccc([N+](=O)[O-])c2nonc12 ; COC(=O)C(CS)NC(=O)C(Cc1ccccc1)NC(=O)C(Cc1ccccc1)NC(=O)CCNc1ccc([N+](=O)[O-])c2nonc12]

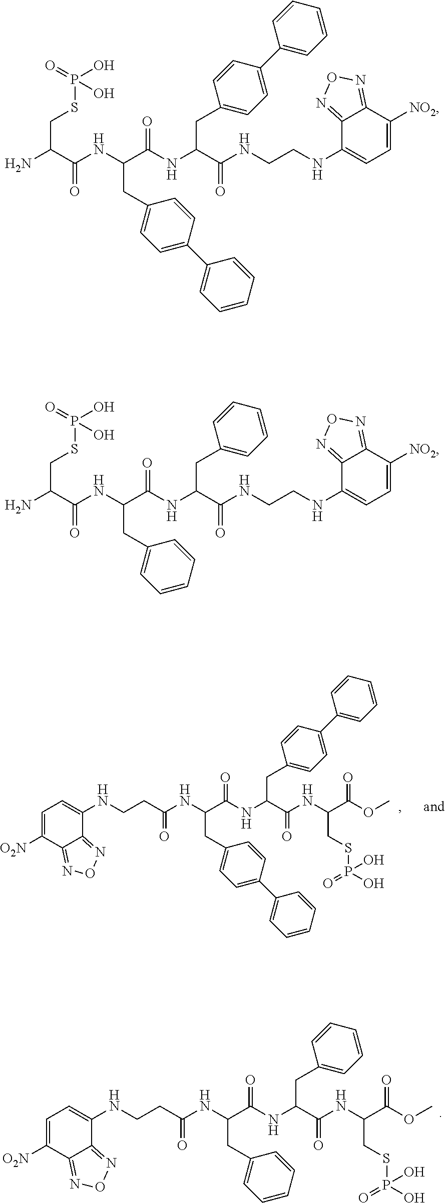 [SMILES: NC(CSP(=O)(O)O)C(=O)NC(Cc1ccc(-c2ccccc2)cc1)C(=O)NC(Cc1ccc(-c2ccccc2)cc1)C(=O)NCCNc1ccc([N+](=O)[O-])c2nonc12 ; NC(CSP(=O)(O)O)C(=O)NC(Cc1ccccc1)C(=O)NC(Cc1ccccc1)C(=O)NCCNc1ccc([N+](=O)[O-])c2nonc12 ; COC(=O)C(CSP(=O)(O)O)NC(=O)C(Cc1ccc(-c2ccccc2)cc1)NC(=O)C(Cc1ccc(-c2ccccc2)cc1)NC(=O)CCNc1ccc([N+](=O)[O-])c2nonc12 ; COC(=O)C(CSP(=O)(O)O)NC(=O)C(Cc1ccccc1)NC(=O)C(Cc1ccccc1)NC(=O)CCNc1ccc([N+](=O)[O-])c2nonc12]

According to one approach, the compounds of the present invention can be synthesized by standard peptide synthesis operations. These include both FMOC (9-fluorenylmethyloxy-carbonyl) and tBoc (tert-butyloxy-carbonyl) synthesis protocols that can be carried out on automated solid phase peptide synthesis instruments including, without limitation, the Applied Biosystems 431 A, 433 A synthesizers and Peptide Technologies Symphony or large-scale Sonata or CEM Liberty automated solid phase peptide synthesizers. The use of alternative peptide synthesis instruments is also contemplated. Peptides prepared using solid phase synthesis are recovered in a substantially pure form.

By way of example, a mercaptoalkanol (e.g., mercaptoethanol, 3-mercapto-1-propanol, 4-mercapto-1-propanol, 5-mercapto-butanol, 6-mercapto-1-hexanol) can be linked to 2-Chlorotrityl chloride resin, and the rest of the amino acid residues can then be coupled using solid phase peptide synthesis. The peptide-resin is cleaved by 95% of TFA and excess amount of the appropriate acyl chloride is added to the thiopeptide solution in TFA with ice/water bath. The reaction is run overnight, and the reaction mixture is concentrated and followed by purification using HPLC or flash column.

Another aspect of the invention relates to a composition that includes one or more compounds of formula (I) or formula (II), as described herein, in an aqueous medium or carrier. Both diagnostic compositions and pharmaceutically acceptable compositions are contemplated; in the latter embodiment the aqueous medium or carrier is a pharmaceutically acceptable carrier containing pharmaceutically acceptable diluents, additives, and the like.

In certain embodiments, the compounds of formula (I) or formula (II) are present in a composition of the invention at a concentration of from about 1 nM to about 500 mM, such as from about 10 nM to about 200 mM, about 50 nM to about 100 mM, about 100 nM to about 10 mM, about 200 nM to about 1 mM, about 300 nM to about 500 μM, about 400 nM to about 500 μM, or about 500 nM to about 500 μM; or from about 50 nM to about 10 μM, about 100 nM to about 10 μM, about 200 nM to about 10 μM, about 300 nM to about 10 μM, or about 500 nM to about 10 μM.

Diagnostic compositions containing the compounds of formula (I) or formula (II) can be used for labeling of the Golgi Apparatus for cellular studies. In such embodiments, one or both of Z₁ or Z₂ of formula (I) and Z₃ or Z₄ of formula (II) is a fluorophore, or the dipeptide of formula (I) or the polypeptide of formula (II) includes a non-natural amino acid containing a fluorophore.

Pharmaceutical compositions may optionally contain from about 0.1 pg to about 1.0 g of the compounds of formula (I) or formula (II), such as from about 10 μg to about 0.5 g, about 100 μg to about 0.1 g (including about 100 μg to about 500 mg, about 100 μg to about 250 mg, or about 100 μg to about 100 mg).

Pharmaceutical compositions may optionally comprise one or more therapeutic agents to be delivered simultaneously with the compounds of formula (I) or formula (II). For example, when used in the treatment of a particular condition, the compounds of formula (I) or formula (II) can be co-administered with another therapeutic agent, as a single formulation containing both active ingredients. Alternatively, a pharmaceutically acceptable formulation containing a therapeutic agent can be administered before, after, or concomitantly with a formulation containing one or more of the compounds of formula (I) or formula (II).

Exemplary therapeutic agents that can be present in the same formulation, or co-administered as separate formulations, include antioxidants, coenzymes, vitamins, metabolites, analgesics, anti-inflammatory agents, antihelminthics, anti-arrhythmic agents, antibacterial agents, anti-viral agents, anti-coagulants, anti-depressants, anti-diabetics, anti-epileptics, anti-fungal agents, anti-gout agents, anti-hypertensive agents, anti-thrombogenic agents, anti-claudication agents, anti-atherosclerotic drugs, vascular agents, anti-malarials, anti-migraine agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, b-blockers, cardiac inotropic agents, corticosteroids, diuretics, anti-parkinsonian agents, gastro-intestinal agents, histamine receptor antagonists, keratolyptics, lipid regulating agents, anti-anginal agents, Cox-2 inhibitors, leukotriene inhibitors, macrolides, muscle relaxants, nutritional agents, opioid analgesics, protease inhibitors, sex hormones, stimulants, anti-osteoporosis agents, anti-obesity agents, cognition enhancers, anti-urinary incontinence agents, anti-benign prostate hypertrophy agents, essential fatty acids, non-essential fatty acids, cytokines, growth factors, antibodies, radioprotective agents, and cardioprotective agents. Further non-limiting examples of the therapeutic agents include: acetretin, albendazole, albuterol, aminoglutethimide, amiodarone, amlodipine, amphetamine, amphotericin B, arginine, atorvastatin, atovaquone, azithromycin, baclofen, beclomethasone, benazepril, benzonatate, betamethasone, bicalutanide, budesonide, bupropion, busulfan, butenafme, calcifediol, calcipotriene, calcitriol, camptothecin, candesartan, capsaicin, captopril, carbamezepine, carotenes, celecoxib, cerivastatin, cetirizine, chlorpheniramine, cholecalciferol, cilazepril, cilostazol, cimetidine, cinnarizine, ciprofloxacin, cisapride, clarithromycin, clemastine, clomiphene, clomipramine, clonidine, clopidogrel, codeine, coenzyme Q10, cyclobenzaprine, cyclosporin, danazol, dantrolene, dexchlorpheniramine, diclofenac, dicoumarol, digoxin, dehydroepiandrosterone, dihydroergotamine, dihydrotachysterol, dirithromycin, donezepil, doxazosin, efavirenz, eprosartan, ergocalciferol, ergotamine, essential fatty acid sources, etodolac, etoposide, famotidine, fenofibrate, fentanyl, fexofenadine, finasteride, fluconazole, flurbiprofen, fluvastatin, fosphenyloin, frovatriptan, fuirazolidone, gabapentin, gemfibrozil, glibenclamide, glipizide, glyburide, glimepiride, griseofulvin, halofantrine, ibuprofen, irbesartan, irinotecan, isosorbide dinitrate, isotretinoin, itraconazole, ivermectin, ketenserin, ketoconazole, ketorolac, lamotrigine, lansoprazole, leflunomide, lisinopril, loperamide, loratadine, losartan, lovastatin, L-thryroxine, lutein, lycopene, medroxyprogesterone, mifepristone, mefloquine, megestrol acetate, methadone, methoxsalen, methyldopa, metronidazole, miconazole, midazolam, miglitol, minoxidil, mitoxantrone, montelukast, moxonidine, nabumetone, nalbuphine, naratriptan, nelfmavir, nifedipine, nil solidipine, nilutanide, nitrofurantoin, nitroglycerin, nizatidine, omeprazole, oprevelkin, oestradiol, oxaprozin, paclitaxel, paracalcitol, paroxetine, pentazocine, pioglitazone, pizofetin, prazosin, pravastatin, prednisolone, probucol, progesterone, pseudoephedrine, pyridostigmine, rabeprazole, raloxifene, rofecoxib, repaglinide, rifabutine, rifapentine, rimexolone, ritanovir, rizatriptan, rosiglitazone, saquinavir, sertraline, sibutramine, sildenafil citrate, simvastatin, sirolimus, spironolactone, sumatriptan, tacrine, tacrolimus, tamoxifen, tamsulosin, targretin, tazarotene, telmisartan, teniposide, terbinafme, terazosin, tetrahydrocannabinol, tiagabine, ticlopidine, tirofibran, tizanidine, topiramate, topotecan, toremitfene, tramadol, tretinoin, troglitazone, trovafloxacin, ubidecarenone, urapidil, valsartan, venlafaxine, verteporfm, vigabatrin, vitamin A, vitamin D, vitamin E, vitamin K, zafirlukast, zileuton, zolmitriptan, zolpidem, zopiclone, pharmaceutically acceptable salts, isomers, and derivatives thereof, and mixtures thereof. Typically, single doses of the therapeutic agent range from 1 μg/kg body weight to 1000 mg/kg body weight (although lesser or greater dosages are also contemplated).

In some embodiments, the carrier is an aqueous medium. In one embodiment, the aqueous medium is a sterile isotonic aqueous buffer, which is typically well tolerated for administration to an individual. Additional exemplary aqueous media include, without limitation, normal saline (about 0.9% NaCl), phosphate buffered saline (“PBS”), sterile water/distilled autoclaved water (“DAW”), as well as cell growth medium (e.g., MEM, with or without serum), aqueous solutions of dimethyl sulfoxide (“DMSO”), polyethylene glycol (“PEG”), and/or dextran (less than 6% per by weight).

To improve patient tolerance to administration, the pharmaceutical composition may have a pH of about 4.5 to about 8 5 In some embodiments, sodium hydroxide or hydrochloric is added to the pharmaceutical composition to adjust the pH.

In other embodiments, the pharmaceutical composition includes a weak acid or salt as a buffering agent to maintain pH. Citric acid has the ability to chelate divalent cations and can thus also prevent oxidation, thereby serving two functions as both a buffering agent and an antioxidant stabilizing agent. Citric acid is typically used in the form of a sodium salt, typically 10-500 mM. Other weak acids or their salts can also be used.

Both diagnostic and pharmaceutical compositions may also include solubilizing agents, preservatives, stabilizers, emulsifiers, and the like. A local anesthetic (e.g., lidocaine, benzocaine, etc.) may also be included in the pharmaceutical compositions, particularly for injectable forms, to ease pain at the site of the injection.

Administration of the pharmaceutical composition can be repeated on a daily schedule (i.e., once, twice, or thrice daily), or according to a periodic schedule (i.e., once weekly, twice weekly, thrice weekly, bimonthly, once monthly).

Individuals that can be treated include both veterinary patients, typically but not exclusively mammals, as well as human patients.

One aspect of the invention relates to a method of delivering a drug moiety into the Golgi apparatus. This method includes providing a compound according to formula (I) or formula (II), wherein Z₂ or Z₄ is a drug moiety, or a composition as described herein, and contacting a cell with the compound or composition, whereby the compound is taken up by the cell and targeted to the Golgi apparatus within the cell. In some embodiments, the drug moiety is hydrophobic. In certain embodiments, the cell is ex vivo. In other embodiments, the cell is in vivo.

Another aspect of the invention relates to a method of treating a patient having a cancerous condition. This method includes administering a pharmaceutical composition as described herein to a patient having a cancerous condition, where the administering is effective to inhibit cancer cell survival. Modes and frequency of administration, and patient groups include those identified above.

The cancerous conditions to be treated in accordance with this aspect can involve cancer cells present in a solid tumor, present as a metastatic cell, or present in a heterogenous population of cells that includes both cancerous and noncancerous cells. Exemplary cancer conditions include, without limitation, cancers or neoplastic disorders of the brain and CNS (glioma, malignant glioma, glioblastoma, astrocytoma, multiforme astrocytic gliomas, medulloblastoma, craniopharyngioma, ependymoma, pinealoma, hemangioblastoma, acoustic neuroma, oligodendroglioma, meningioma), pituitary gland, breast (Infiltrating, Pre-invasive, inflammatory cancers, Paget's Disease, Metastatic and Recurrent Breast Cancer), blood (Hodgkin's Disease, Leukemia, Multiple Myeloma, Lymphoma), lymph node cancer, lung (Adenocarcinoma, Oat Cell, Non-small Cell, Small Cell, Squamous Cell, Mesothelioma), skin (melanoma, basal cell, squamous cell, Kaposi's Sarcoma), bone cancer (Ewing's Sarcoma, Osteosarcoma, Chondrosarcoma), head and neck (laryngeal, pharyngeal, and esophageal cancers), oral (jaw, salivary gland, throat, thyroid, tongue, and tonsil cancers), eye, gynecological (Cervical, Endometrial, Fallopian, Ovarian, Uterine, Vaginal, and Vulvar), genitourinary (Adrenal, bladder, kidney, penile, prostate, testicular, and urinary cancers), and gastrointestinal (appendix, bile duct (extrahepatic bile duct), colon, gallbladder, gastric, intestinal, liver, pancreatic, rectal, and stomach cancers).

In this aspect of the invention, the pharmaceutical composition may contain the depsipeptide thioester compound(s), alone, or in combination with a cancer therapeutic agent of the type described above.

While any class of antineoplastic agent, anticancer drug, or chemotherapeutic drug is contemplated for use in connection with the present invention, exemplary agents within these classes include alkylating agents, platinum drugs, antimetabolites, anthracycline and nonanthracycline antitumor antibiotics, topoisomerase inhibitors, mitotic inhibitors, corticosteroids, and targeted immunotherapies (such as imatinib, Gleevec®; gefitinib, Iressa®; sunitinib, Sutent®; and bortezomib, Velcade®).

Yet another aspect of the invention relates to a method of imaging a cell. The method includes providing a compound according to formula (I) or formula (II), or a composition as described herein, wherein Z₁ or Z₂ of formula (I) or Z₃ or Z₄ of formula (II) is a fluorophore, or the compound of formula (I) or formula (II) includes a non-natural amino acid containing a fluorophore moiety, and then contacting a cell with the compound or the composition, whereby the compound is taken up by the cell and targeted to the Golgi apparatus within the cell. Thereafter, an image of the cell can be obtained using any desired image capture equipment or techniques compatible with the fluorophore moiety that is present within the compound. In the image, the Golgi apparatus is identified by fluorescence from the fluorophore.

As used herein, the term “about” when used in connection with a numerical value denotes an interval of accuracy that is ±10% in certain embodiments, ±5% in other embodiments, ±2.5% in still further embodiments, and ±1% in yet another embodiment.

### EXAMPLES

The examples below are intended to exemplify the practice of embodiments of the disclosure but are by no means intended to limit the scope thereof.

### Materials and Methods for Examples 1-3

2-Cl-trityl chloride resin (1.0 mmol/g), Fmoc protected amino acid, and HBTU were obtained from GL Biochem (Shanghai, China). N, N-diisopropylethylamine (DIEA) and solvents were obtained from Fisher Scientific. Acetyl chloride and mercaptoethanol were purchased from TCI America. All the chemical reagents and solvents were used as received from commercial sources without further purification. Minimum Essential Media (MEM), Dulbecco's Modified Eagle Medium (DMEM), McCoy's 5A Medium, and RPMI-1640 Medium were purchased from ATCC. Fetal bovine serum (FBS) and Penicillin-Streptomycin from Gibco by Life Technologies. All precursors and compounds were purified by a reverse phase HPLC (Agilent 1100 Series) equipped with an XTerra C18 RP column, and HPLC grade acetonitrile (0.1% TFA) and HPLC grade water (0.1% TFA) were used as the eluents. The LC-MS spectra were obtained with a Waters Acquity Ultra Performance LC with Waters Micromass detector. Transmission electron microscope (TEM) images were obtained on Morgagni 268 transmission electron microscope. Fluorescence images were taken by ZEISS LSM 880 confocal laser scanning microscope and ZEISS LSM 880 AiryScan Fast Confocal System.

### Example 1—Synthesis of Peptides 1—4

Peptide Thioesters 1 and 2 were synthesized according to Scheme 1 below:

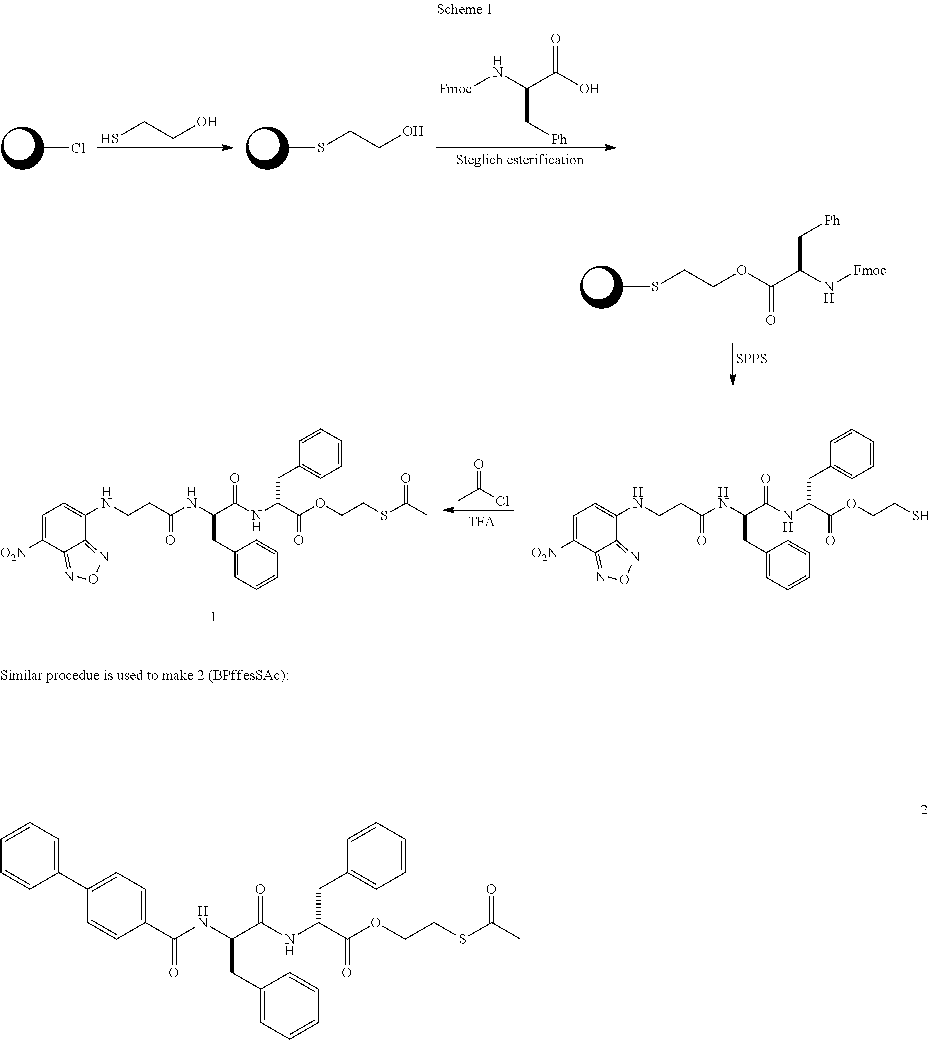

Standard Fmoc chemistry solid phase peptide synthesis was used utilizing 2-chlorotrityl chloride resin and the corresponding Fmoc-protected amino acids with side chains properly protected. Briefly, the 2-Cl resin (1 g) was swelling in dry DCM for 30 min and then 20.0 equiv. of mercaptoethanol in dry DMF was loaded onto the resin and the reaction was run overnight. Then, the first amino acid was coupled to the hydroxyl group via Steglich esterification. After loading the first amino acid to the resin, Fmoc group was removed with 20% piperidine in DMF, the next Fmoc-protected amino acid was coupled to the free amino group using HBTU as the coupling reagent. The peptide chain was cleaved from the resin by 95% TFA (95% TFA, 2.5% TIPS, 2.5% H₂O) for 1 h. The Depsipeptide thiols in TFA solution was transferred into a 100-mL round bottom flask, and the corresponding acyl chloride (20 equiv.) was added dropwise into the solution with an iced water bath. The reaction was run overnight followed by the removal of TFA and the excessive acyl chloride by a rotary evaporator. The resulting oily products were purified with HPLC and then dried by a lyophilizer.

Peptide 3 was synthesized using solid phase peptide synthesis according to Scheme 2 below:

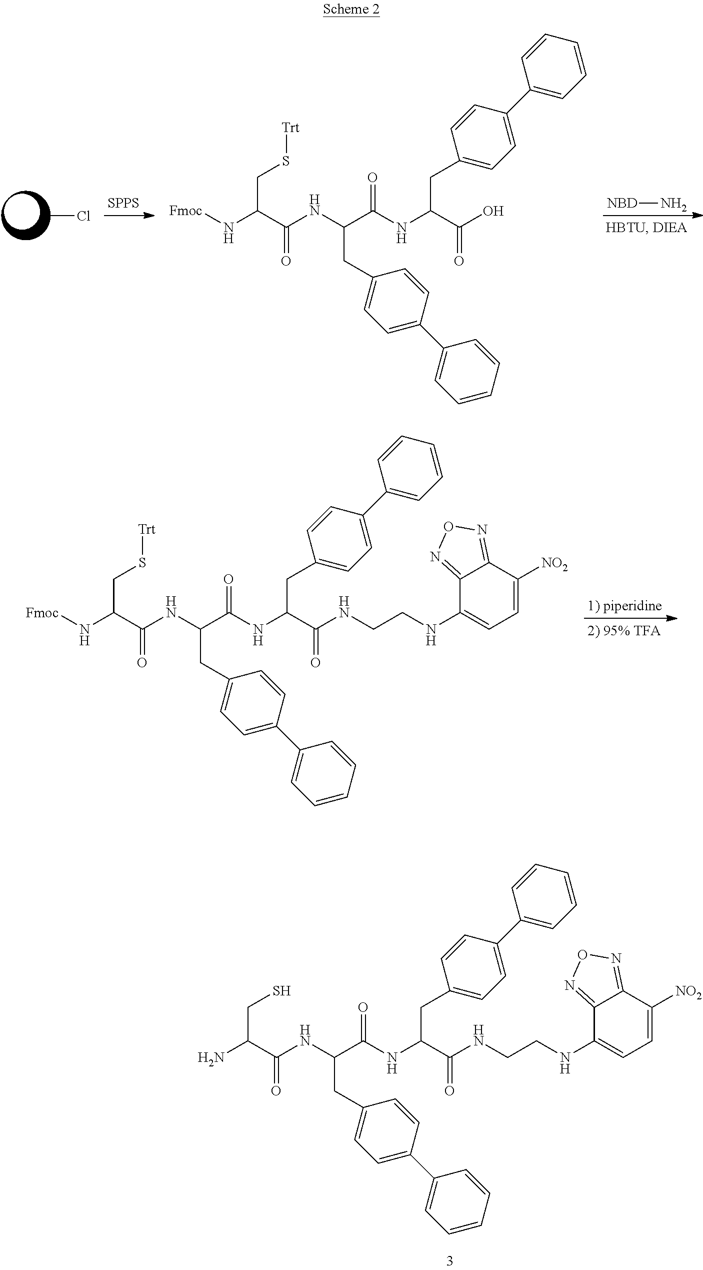

Peptide 4 was synthesized using solid phase peptide synthesis according to Scheme 3 below:

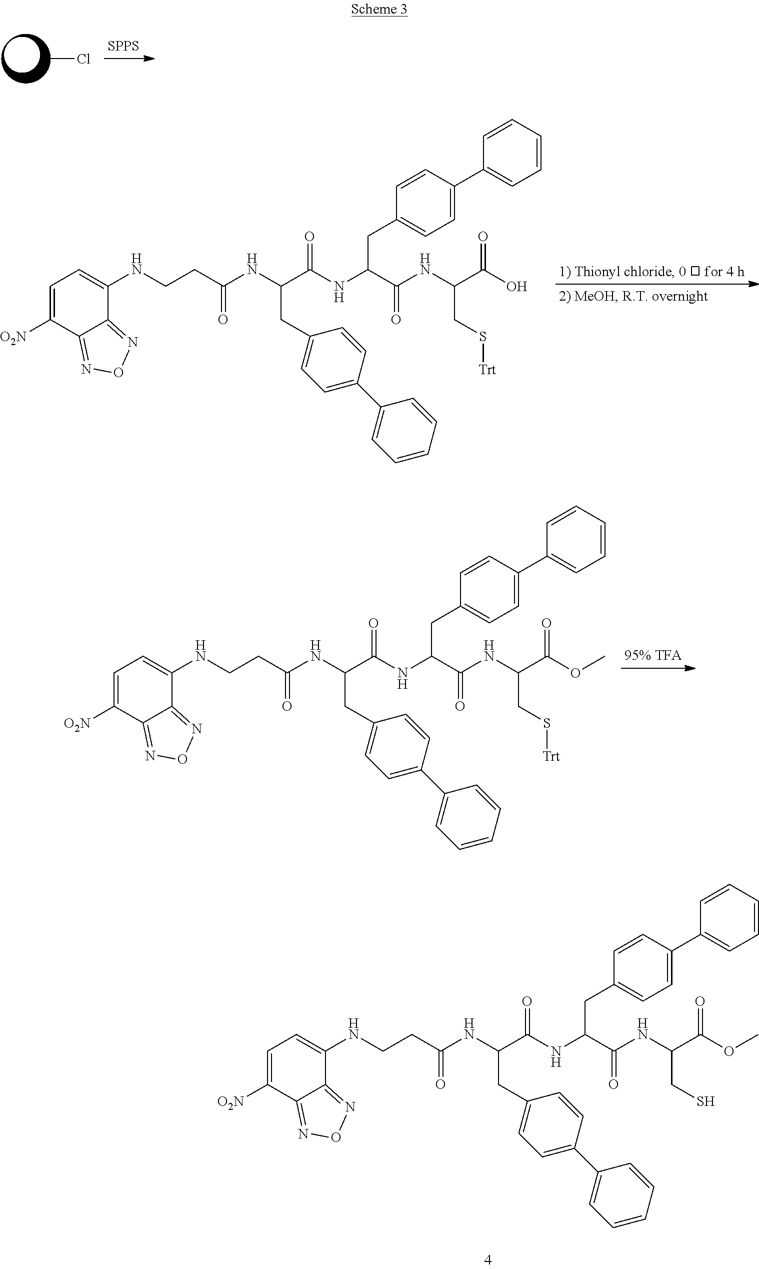

### Example 2—Imaging Cells with Peptide Thioester 1

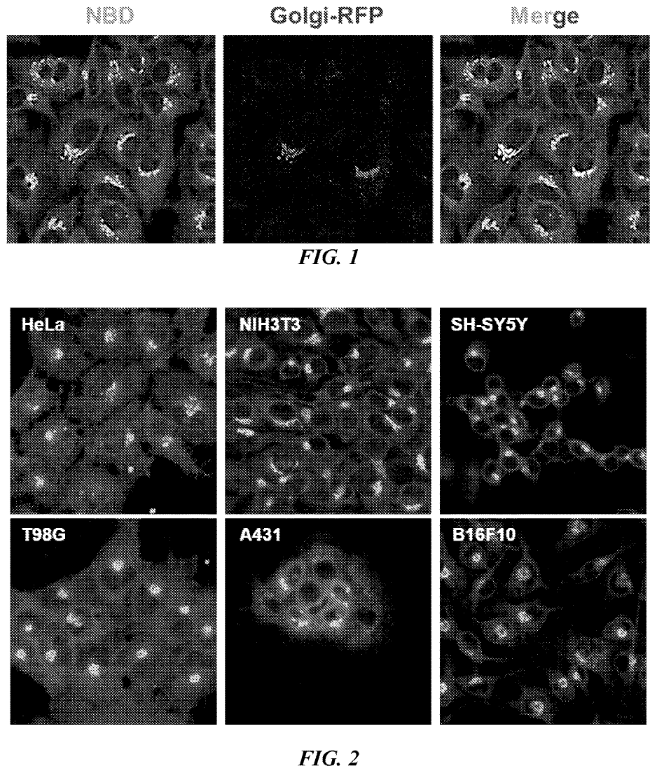

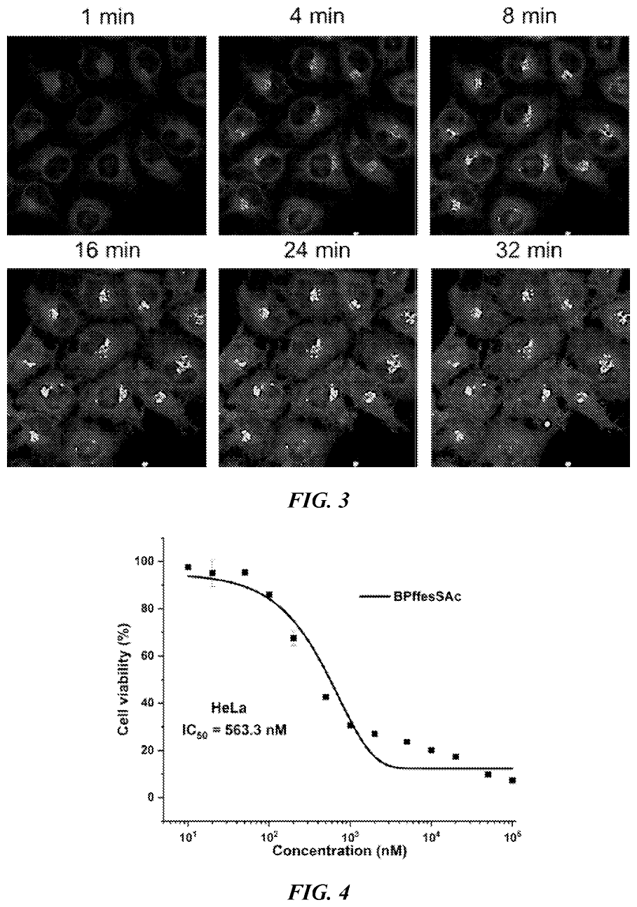

The treatment of GALNT1-RFP transfected HeLa cells with compound 1 (1 M, 10 min) results in a perfect overlap of green and red fluorescence (FIG. 1), indicating compound 1 specifically locates at Golgi apparatus of HeLa cells. 1, acting as a universal Golgi probe, can accumulate at Golgi of various cell lines, including HeLa, NIH3T3, SH-SY5Y, T98G, A431, B16F10, etc. (FIG. 2). Treating cells with a low concentration (i.e., 500 nM) of 1 also leads to a fluorescence build-up at Golgi in a short period of time (FIG. 3), suggests a supreme Golgi targeting ability of peptide thioester 1.

### Example 3—Cytotoxicity of Peptide Thioester 2 Against Cancer Cells

Cancer immunotherapy has shown great promise in treating cancer, but currently only a small percentage of patients respond to these treatments. Therefore, there is a need for new strategies to overcome resistance to cancer immunotherapy. The in-situ formed nanoparticles from 2 can selectively disrupt the dynamics of the Golgi apparatus in cancer cells. These Golgi-disrupting nanoparticles (GNPs) from 2 will inhibit immunoresistant cancer cells as monotherapy or as combination therapy for boosting cancer immunotherapy. The broad application of this type of molecules is to serve a first-in-kind nanomedicine to overcome immunoresistant or acquired drug resistance in cancer therapy, with the aim of reducing the morbidity associated with cancer.

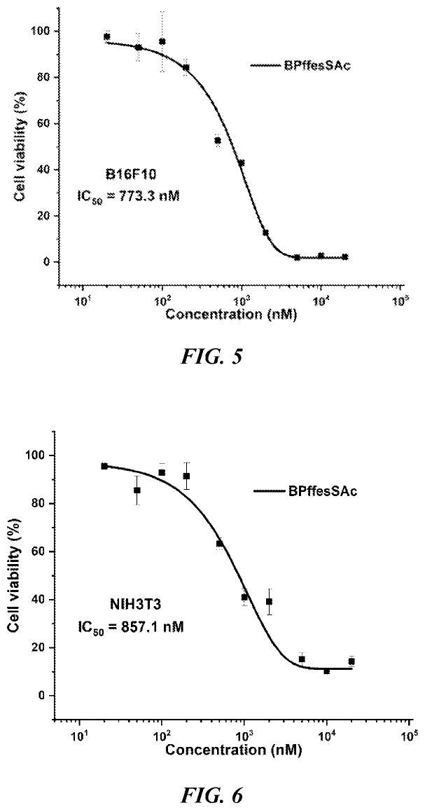

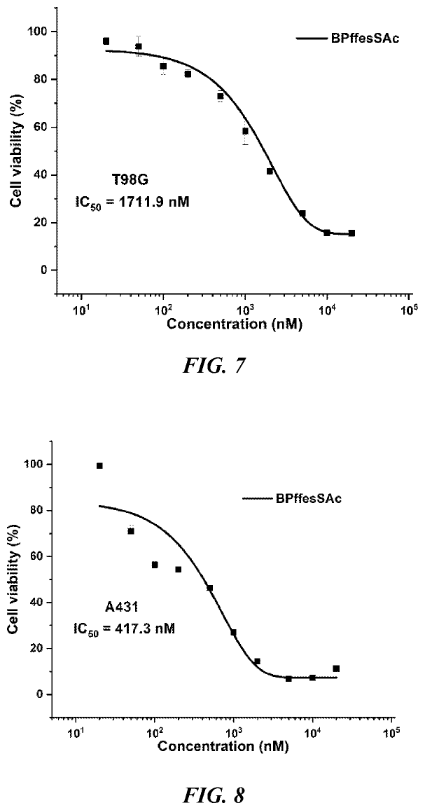

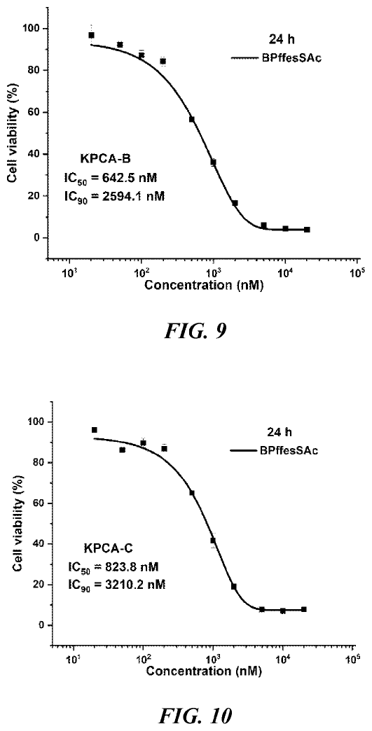

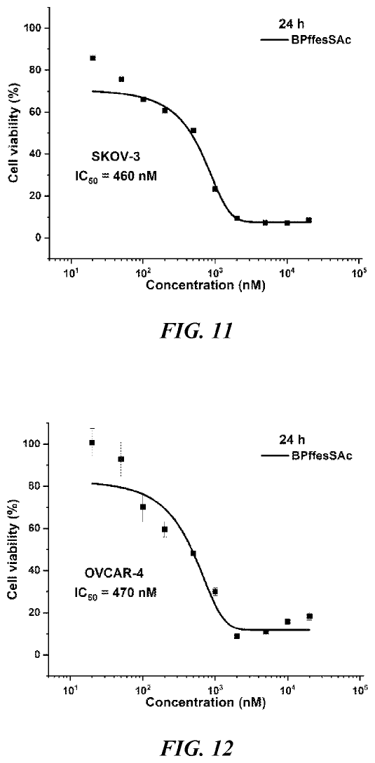

Peptide thioester 2 was shown to be able to inhibit cancer cells effectively even at sub-micromolar range. The IC₅₀ of 2 against HeLa, B16F10, NIH3 T3, T98G, A-431, KPCA-B, KPCA-C, SKOV-3 and OVCAR-4 is about 0.56 M, 0.77 M, 0.86 M, 1.7 M, 0.42 M, 0.64 M, 0.82 M, 0.46 M and 0.47 M, respectively (FIGS. 4-12). The in vivo antitumor activity of 2 will be evaluated as well, e.g., using a xenograft model of disease.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A compound according to formula (I) 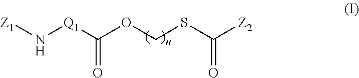
where
Z₁ is an aromatic-containing group, preferably an arylacyl;
NH-Q-C(O) is a dipeptide containing natural or unnatural amino acids, which can be the same or different;
n is an integer from 1 to 12, such as 1 to 6, preferably 1 or 2;
Z₂ is a C₁-C₆ alkyl, C₁-C₆ alkenyl, C₁-C₆ alkynyl, —N(CH₃)₂, —CH₂OC(O)CH₃, —CH₂C(O)OCH₃, 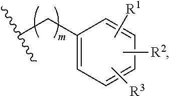
a therapeutic agent (drug moiety), or a fluorophore;
R¹, R², and R³ are independently selected at each occurrence from the group consisting of H, halogen, C₁₋₄ alkyl, —OC₁₋₄ alkyl, and —NO₂; and
m is 0, 1, 2, 3, 4, 5, or 6.

2. The compound according to claim 1, wherein Z₁ is an arylacyl selected from the group of

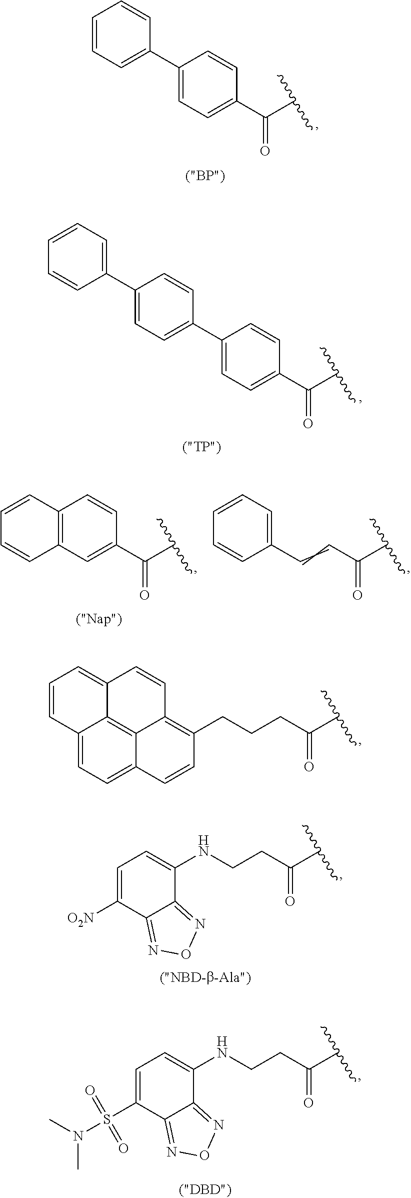

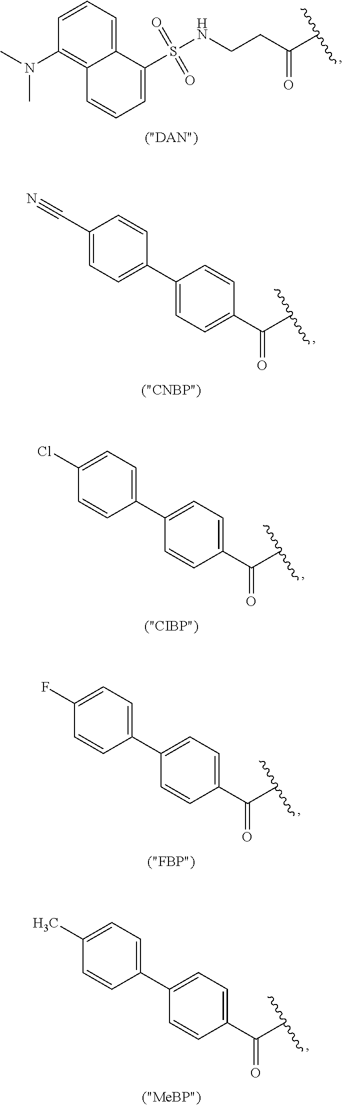 [SMILES: *C(=O)CCNS(=O)(=O)c1cccc2c(N(C)C)cccc12 ; *C(=O)c1ccc(-c2ccc(C#N)cc2)cc1 ; *C(=O)c1ccc(-c2ccc(Cl)cc2)cc1 ; *C(=O)c1ccc(-c2ccc(F)cc2)cc1 ; *C(=O)c1ccc(-c2ccc(C)cc2)cc1]

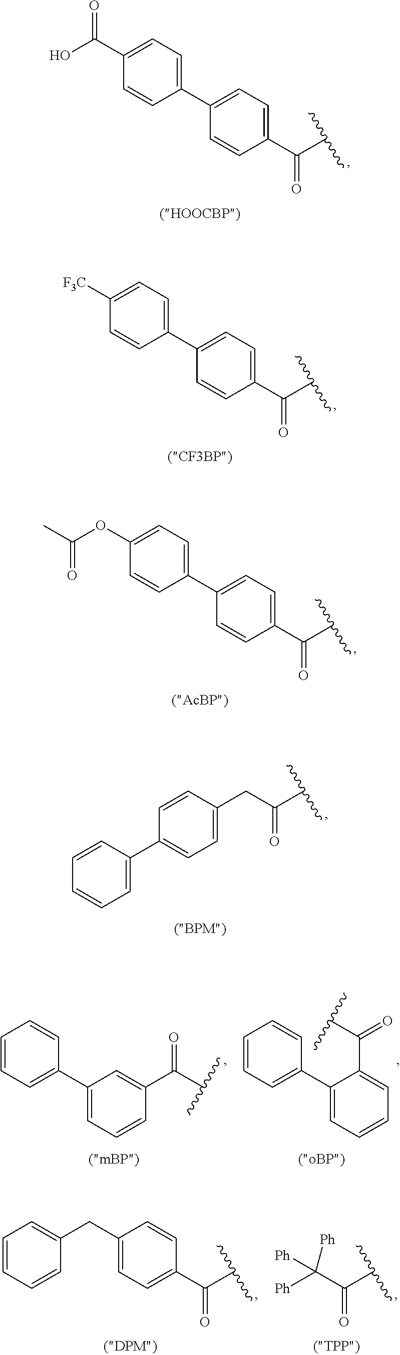

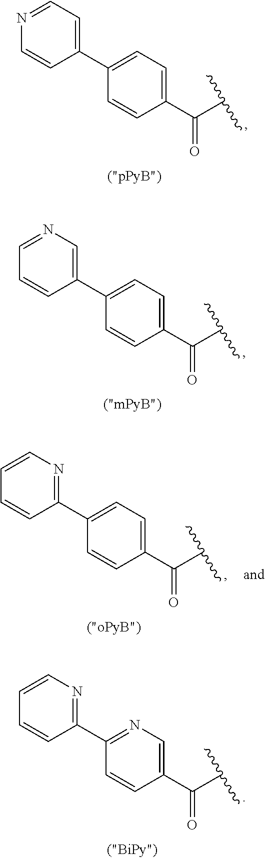 [SMILES: *C(=O)c1ccc(-c2ccncc2)cc1 ; *C(=O)c1ccc(-c2cccnc2)cc1 ; *C(=O)c1ccc(-c2ccccn2)cc1 ; *C(=O)c1ccc(-c2ccccn2)nc1]

3-6. (canceled)

7. The compound according to claim 1, wherein the dipeptide NH-Q-C(O) comprises two amino acid residues selected from the group of Phe, Val, Gly, Leu, Met, Tyr, Ile, Ala, Leu, Nal, Dmt, I-Phe, and dap(NBD).

8. The compound according to claim 1, wherein the dipeptide NH-Q-C(O) comprises two amino acid residues independently selected from the group of D-Phe, D-Val, Gly, D-Leu, D-Met, D-Tyr, D-Ile, D-Ala, D-Nal, D-Leu, L-Leu, L-photo-Met, I-Phe, and dap(NBD).

9.-18. (canceled)

19. The compound according to claim 2, wherein the compound of formula (I) is selected from the group of: 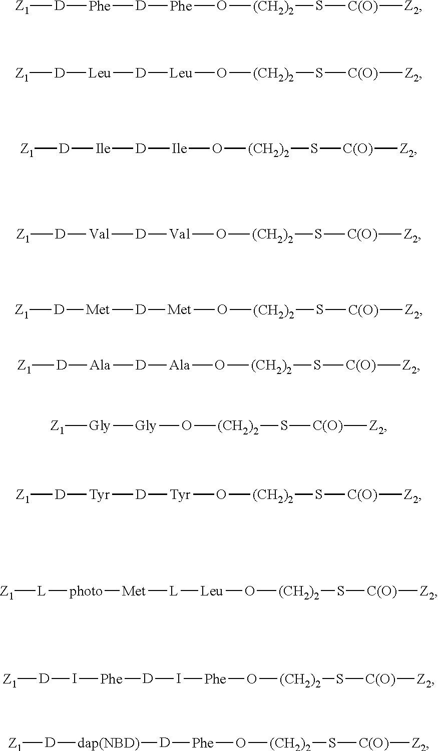
where Z₂ is —CH₃, —CH₂CH₃, —N(CH₃)₂, —CH₂OC(O)CH₃, —CH₂C(O)OCH₃, or phenyl.

20. A composition comprising the compound according to claim 1 in an aqueous medium.

21. The composition according to claim 20, wherein the compound is present in the form of nanoparticles.

22. A pharmaceutical composition comprising the compound according to claim 1 in a pharmaceutically acceptable carrier.

23. The pharmaceutical composition according to claim 22, wherein the composition is in the form of an injectable solution or suspension.

24. A method of delivering a drug moiety into the Golgi apparatus comprising:
providing a compound according to claim 1, wherein Z₂ is a drug moiety, or a composition comprising the compound; and
contacting a cell with the compound or the composition, whereby the compound is taken up by the cell and targeted to the Golgi apparatus within the cell.

25.-27. (canceled)

28. A method of treating a patient having a cancerous condition comprising:
administering a pharmaceutical composition according to claim 22 to a patient having a cancerous condition, wherein said administering is effective to inhibit cancer cell survival.

29.-30. (canceled)

31. A method of imaging a cell, the method comprising:
providing a compound according to claim 1, wherein Z₁ or Z₂ comprises a fluorophore or the dipeptide comprises a non-natural amino acid comprising a fluorophore moiety, or a composition comprising the compound;
contacting a cell with the compound or the composition, whereby the compound is taken up by the cell and targeted to the Golgi apparatus within the cell; and
obtaining an image of the cell, whereby the Golgi apparatus is identified by fluorescence from the fluorophore.

32. A compound according to formula (II) 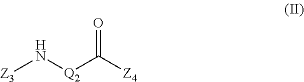
where
Z₃ is H or an aromatic-containing group, preferably an arylacyl;
NH-Q₂-C(O) is a peptide containing at least three amino acid residues, including at least two aromatic amino acid residues and at least one cysteine residue, wherein the cysteine residue is optionally a modified cysteine residue having a phosphate (—PO₃H₂) group or the group —C(O)(CH₂)ₚCH₃, where p is an integer from 0 to 5, attached to the sidechain sulfur group;
Z₄ is —O(CH₂)_{q}CH₃ where q is an integer from 0 to 5, a therapeutic agent (drug moiety), or a fluorophore, optionally with the group —NH(CH₂)ᵣNH— or —NH(CH₂)ᵣC(O)— between the therapeutic agent or fluorophore and the C-terminal amino acid group, where r is an integer from 0 to 5.

33.-40. (canceled)

41. The compound according to claim 32, wherein the compound of formula (II) is selected from the group of:
Z₃-L-(S-acetyl)Cys-L-bPhe-L-bPhe-Z₄,
Z₃-D-(S-acetyl)Cys-D-bPhe-D-bPhe-Z₄,
Z₃-L-(S-acetyl)Cys-L-Phe-L-Phe-Z₄,
Z₃-D-(S-acetyl)Cys-D-Phe-D-Phe-Z₄,
Z₃-L-(S-acetyl)Cys-L-Tyr-L-Tyr-Z₄,
Z₃-D-(S-acetyl)Cys-D-Tyr-D-Tyr-Z₄,
Z₃-L-(S-acetyl)Cys-L-(1-Nal)-L-(1-Nal)-Z₄,
Z₃-D-(S-acetyl)Cys-D-(1-Nal)-D-(1-Nal)-Z₄,
Z₃-L-(S-acetyl)Cys-L-(2-Nal)-L-(2-Nal)-Z₄,
Z₃-D-(S-acetyl)Cys-D-(2-Nal)-D-(2-Nal)-Z₄,
Z₃-L-(S-acetyl)Cys-L-(4-(2-phenylethyl)Phe-L-(4-(2-phenylethyl)Phe-Z₄,
Z₃-D-(S-acetyl)Cys-D-(4-(2-phenylethyl)Phe-D-(4-(2-phenylethyl)Phe-Z₄,
Z₃-L-(S-acetyl)Cys-L-(4-(2-phenylethenyl)Phe-L-(4-(2-phenylethenyl)Phe-Z₄,
Z₃-D-(S-acetyl)Cys-D-(4-(2-phenylethenyl)Phe-D-(4-(2-phenylethenyl)Phe-Z₄,
Z₃-L-(S—PO₃H₂)Cys-L-bPhe-L-bPhe-Z₄,
Z₃-D-(S—PO₃H₂)Cys-D-bPhe-D-bPhe-Z₄,
Z₃-L-(S—PO₃H₂)Cys-L-Phe-L-Phe-Z₄,
Z₃-D-(S—PO₃H₂)Cys-D-Phe-D-Phe-Z₄,
Z₃-L-(S—PO₃H₂)Cys-L-Tyr-L-Tyr-Z₄,
Z₃-D-(S—PO₃H₂)Cys-D-Tyr-D-Tyr-Z₄,
Z₃-L-(S—PO₃H₂)Cys-L-(1-Nal)-L-(1-Nal)-Z₄,
Z₃-D-(S—PO₃H₂)Cys-D-(1-Nal)-D-(1-Nal)-Z₄,
Z₃-L-(S—PO₃H₂)Cys-L-(2-Nal)-L-(2-Nal)-Z₄,
Z₃-D-(S—PO₃H₂)Cys-D-(2-Nal)-D-(2-Nal)-Z₄,
Z₃-L-(S—PO₃H₂)Cys-L-(4-(2-phenylethyl)Phe-L-(4-(2-phenylethyl)Phe-Z₄,
Z₃-D-(S—PO₃H₂)Cys-D-(4-(2-phenylethyl)Phe-D-(4-(2-phenylethyl)Phe-Z₄,
Z₃-L-(S—PO₃H₂)Cys-L-(4-(2-phenylethenyl)Phe-L-(4-(2-phenylethenyl)Phe-Z₄,
Z₃-D-(S—PO₃H₂)Cys-D-(4-(2-phenylethenyl)Phe-D-(4-(2-phenylethenyl)Phe-Z₄,
Z₃-L-Cys-L-bPhe-L-bPhe-Z₄,
Z₃-D-Cys-D-bPhe-D-bPhe-Z₄,
Z₃-L-Cys-L-Phe-L-Phe-Z₄,
Z₃-D-Cys-D-Phe-D-Phe-Z₄,
Z₃-L-Cys-L-Tyr-L-Tyr-Z₄,
Z₃-D-Cys-D-Tyr-D-Tyr-Z₄,
Z₃-L-Cys-L-(1-Nal)-L-(1-Nal)-Z₄,
Z₃-D-Cys-D-(1-Nal)-D-(1-Nal)-Z₄,
Z₃-L-Cys-L-(2-Nal)-L-(2-Nal)-Z₄,
Z₃-D-Cys-D-(2-Nal)-D-(2-Nal)-Z₄,
Z₃-L-Cys-L-(4-(2-phenylethyl)Phe-L-(4-(2-phenylethyl)Phe-Z₄,
Z₃-D-Cys-D-(4-(2-phenylethyl)Phe-D-(4-(2-phenylethyl)Phe-Z₄,
Z₃-L-Cys-L-(4-(2-phenylethenyl)Phe-L-(4-(2-phenylethenyl)Phe-Z₄,
Z₃-D-Cys-D-(4-(2-phenylethenyl)Phe-D-(4-(2-phenylethenyl)Phe-Z₄,
Z₃-L-bPhe-L-bPhe-L-(S-acetyl)Cys-Z₄,
Z₃-D-bPhe-D-bPhe-D-(S-acetyl)Cys-Z₄,
Z₃-L-Phe-L-Phe-L-(S-acetyl)Cys-Z₄,
Z₃-D-Phe-D-Phe-D-(S-acetyl)Cys-Z₄,
Z₃-L-Tyr-L-Tyr-L-(S-acetyl)Cys-Z₄,
Z₃-D-Tyr-D-Tyr-D-(S-acetyl)Cys-Z₄,
Z₃-L-(1-Nal)-L-(1-Nal)-L-(S-acetyl)Cys-Z₄,
Z₃-D-(1-Nal)-D-(1-Nal)-D-(S-acetyl)Cys-Z₄,
Z₃-L-(2-Nal)-L-(2-Nal)-L-(S-acetyl)Cys-Z₄,
Z₃-D-(2-Nal)-D-(2-Nal)-D-(S-acetyl)Cys-Z₄,
Z₃-L-(4-(2-phenylethyl)Phe-L-(4-(2-phenylethyl)Phe-L-(S-acetyl)Cys-Z₄,
Z₃-D-(4-(2-phenylethyl)Phe-D-(4-(2-phenylethyl)Phe-D-(S-acetyl)Cys-Z₄,
Z₃-L-(4-(2-phenylethenyl)Phe-L-(4-(2-phenylethenyl)Phe-L-(S-acetyl)Cys-Z₄,
Z₃-D-(4-(2-phenylethenyl)Phe-D-(4-(2-phenylethenyl)Phe-D-(S-acetyl)Cys-Z₄,
Z₃-L-bPhe-L-bPhe-L-Cys-Z₄,
Z₃-D-bPhe-D-bPhe-D-Cys-Z₄,
Z₃-L-Phe-L-Phe-L-Cys-Z₄,
Z₃-D-Phe-D-Phe-D-Cys-Z₄,
Z₃-L-Tyr-L-Tyr-L-Cys-Z₄,
Z₃-D-Tyr-D-Tyr-D-Cys-Z₄,
Z₃-L-(1-Nal)-L-(1-Nal)-L-Cys-Z₄,
Z₃-D-(1-Nal)-D-(1-Nal)-D-Cys-Z₄,
Z₃-L-(2-Nal)-L-(2-Nal)-L-Cys-Z₄,
Z₃-D-(2-Nal)-D-(2-Nal)-D-Cys-Z₄,
Z₃-L-(4-(2-phenylethyl)Phe-L-(4-(2-phenylethyl)Phe-L-Cys-Z₄,
Z₃-D-(4-(2-phenylethyl)Phe-D-(4-(2-phenylethyl)Phe-D-Cys-Z₄,
Z₃-L-(4-(2-phenylethenyl)Phe-L-(4-(2-phenylethenyl)Phe-L-Cys-Z₄,
Z₃-D-(4-(2-phenylethenyl)Phe-D-(4-(2-phenylethenyl)Phe-D-Cys-Z₄,
Z₃-L-bPhe-L-bPhe-L-(S—PO₃H₂)Cys-Z₄,
Z₃-D-bPhe-D-bPhe-D-(S—PO₃H₂)Cys-Z₄,
Z₃-L-Phe-L-Phe-L-(S—PO₃H₂)Cys-Z₄,
Z₃-D-Phe-D-Phe-D-(S—PO₃H₂)Cys-Z₄,
Z₃-L-Tyr-L-Tyr-L-(S—PO₃H₂)Cys-Z₄,
Z₃-D-Tyr-D-Tyr-D-(S—PO₃H₂)Cys-Z₄,
Z₃-L-(1-Nal)-L-(1-Nal)-L-(S—PO₃H₂)Cys-Z₄,
Z₃-D-(1-Nal)-D-(1-Nal)-D-(S—PO₃H₂)Cys-Z₄,
Z₃-L-(2-Nal)-L-(2-Nal)-L-(S—PO₃H₂)Cys-Z₄,
Z₃-D-(2-Nal)-D-(2-Nal)-D-(S—PO₃H₂)Cys-Z₄,
Z₃-L-(4-(2-phenylethyl)Phe-L-(4-(2-phenylethyl)Phe-L-(S—PO₃H₂)Cys-Z₄,
Z₃-D-(4-(2-phenylethyl)Phe-D-(4-(2-phenylethyl)Phe-D-(S—PO₃H₂)Cys-Z₄,
Z₃-L-(4-(2-phenylethenyl)Phe-L-(4-(2-phenylethenyl)Phe-L-(S—PO₃H₂)Cys-Z₄, or
Z₃-D-(4-(2-phenylethenyl)Phe-D-(4-(2-phenylethenyl)Phe-D-(S—PO₃H₂)Cys-Z₄,
where Z₃ is independently H— or NBD-β-Ala, and Z₄ is independently —OCH₃ or —NH(CH₂)₂-NBD.

42. The compound according to claim 32, wherein the compound is selected from the group of

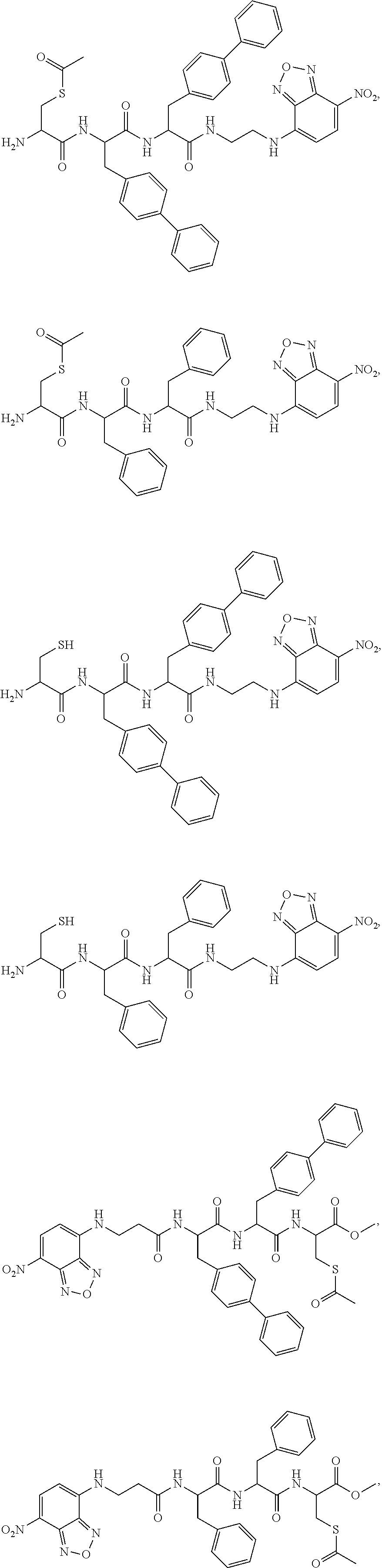

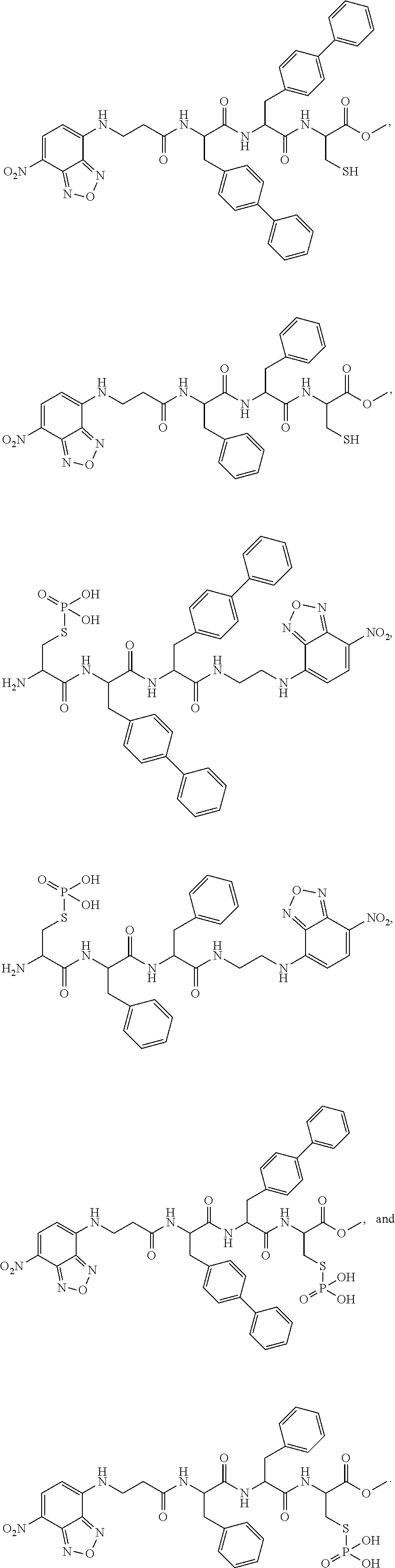

43. A composition comprising the compound according to claim 32 in an aqueous medium.

44. (canceled)

45. A pharmaceutical composition comprising the compound according to claim 32 in a pharmaceutically acceptable carrier.

46. (canceled)

47. A method of delivering a drug moiety into the Golgi apparatus comprising:
providing a compound according to claim 32, wherein Z₄ is a drug moiety, or a composition comprising the compound; and
contacting a cell with the compound or the composition, whereby the compound is taken up by the cell and targeted to the Golgi apparatus within the cell.

48.-50. (canceled)

51. A method of treating a patient having a cancerous condition comprising:
administering a pharmaceutical composition according to claim 45 to a patient having a cancerous condition, wherein said administering is effective to inhibit cancer cell survival.

52.-53. (canceled)
